SEQUENCE LISTING (30 sequences)

SEQ ID NO: 1 - protein, 312 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 2 - DNA, 936 bp, Homo sapiens
atggcaaataatgatgctgttctgaagagactggagcagaagggtgcagaggcagatcaaatcattgaatatcttaagcagcaagtttctctacttaaggagaaagcaattttgcaggcaactttgagggaagagaagaaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggcctggtgaatcatgttcctcttgaacagatgcaaaatcggatggtgattttactttgtaacctgaaacctgcaaagatgaggggagtattatctcaagcaatggtcatgtgtgctagttcaccagagaaaattgaaatcttggctcctccaaatgggtctgttcctggagacagaattacttttgatgctttcccaggagagcctgacaaggagctgaatcctaagaagaagatttgggagcagatccagcctgatcttcacactaatgatgagtgtgtggctacatacaaaggagttccctttgaggtgaaagggaagggagtatgtagggctcaaaccatgagcaacagtggaatcaaa

SEQ ID NO: 3 - protein, 53 aa, Homo sapiens
MANNDAVLKRLEQKGAEADQIIEYLKQQVSLLKEKAILQATLREEKKLRVENA

SEQ ID NO: 4 - protein, 139 aa, Homo sapiens
KLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 5 - DNA, 417 bp, Homo sapiens
aaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 6 - protein, 120 aa, Homo sapiens
LVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 7 - DNA, 35 bp, Artificial Sequence
  AIMP1-(1-53) sense primer
cggaattcatggcaaataatgatgctgttctgaag

SEQ ID NO: 8 - DNA, 33 bp, Artificial Sequence
  AIMP1-(1-53) anti-sense primer
gtctcgagttaagcattttcaactcgaagtttc

SEQ ID NO: 9 - DNA, 35 bp, Artificial Sequence
  AIMP1-(54-192) sense primer
cggaattcaaactgaagaaagaaattgaagaactg

SEQ ID NO: 10 - DNA, 32 bp, Artificial Sequence
  AIMP1-(54-192) anti-sense primer
gtctcgagttagccactgacaactgtccttgg

SEQ ID NO: 11 - DNA, 33 bp, Artificial Sequence
  AIMP1-(193-312) sense primer
cggaattcctggtgaatcatgttcctcttgaac

SEQ ID NO: 12 - DNA, 33 bp, Artificial Sequence
  AIMP1-(193-312) anti-sense primer
gtctcgagttatttgattccactgttgctcatg

SEQ ID NO: 13 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 14 - DNA, 2409 bp, Homo sapiens
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattg

SEQ ID NO: 15 - protein, 266 aa, Homo sapiens
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTE

SEQ ID NO: 16 - protein, 81 aa, Homo sapiens
TVEEPMEEEEAAKEEKEESDDEAAVEEEEEEKKPKTKKVEKTVWDWELMNDIKPIWQRPSKEVEEDEYKAFYKSFSKESDD

SEQ ID NO: 17 - protein, 330 aa, Homo sapiens
PMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDML

SEQ ID NO: 18 - protein, 101 aa, Homo sapiens
RRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAE

SEQ ID NO: 19 - DNA, 303 bp, Homo sapiens
cgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaa

SEQ ID NO: 20 - DNA, 36 bp, Artificial Sequence
  gp96-(22-287) sense primer
gccgaattcgatggacgatgaagttgatgtggatgg

SEQ ID NO: 21 - DNA, 33 bp, Artificial Sequence
  gp96-(22-287) anti-sense primer
cttgtcgacttattcagtcttgctgctccatac

SEQ ID NO: 22 - DNA, 35 bp, Artificial Sequence
  gp96-(288-368) sense primer
gccgaattcgatgactgttgaggagcccatggagg

SEQ ID NO: 23 - DNA, 39 bp, Artificial Sequence
  gp96-(288-368) anti-sense primer
cttgtcgacttagtcatcactttcctttgaaaatgattg

SEQ ID NO: 24 - DNA, 38 bp, Artificial Sequence
  gp96-(369-698) sense primer
gccgaattcgatgcccatggcttatattcactttactg

SEQ ID NO: 25 - DNA, 33 bp, Artificial Sequence
  gp96-(369-698) anti-sense primer
cttgtcgacttacatgtctctgatcagcgggtg

SEQ ID NO: 26 - DNA, 38 bp, Artificial Sequence
  gp96-(699-799) sense primer
gccgaattcgatgcttcgacgaattaaggaagatgaag

SEQ ID NO: 27 - DNA, 35 bp, Artificial Sequence
  gp96-(699-799) anti-sense primer
cttgtcgacttattcagctgtagattcctttgctg

SEQ ID NO: 28 - DNA, 36 bp, Artificial Sequence; identical to SEQ ID NO: 20
  gp96-(288-799) sense primer

SEQ ID NO: 29 - DNA, 35 bp, Artificial Sequence; identical to SEQ ID NO: 27
  gp96-(288-799) anti-sense primer

SEQ ID NO: 30 - DNA, 72 bp, Artificial Sequence
  gp96-(E791) anti-sense primer
cttgtcgacttattcagctgtagattcctttgctgtttcttcttcatctgttcccacatccatttcttcatc

(19) United States
(12) Patent Application Publication
(10) Pub. No.: US 2010/0138941 A1
(43) Pub. Date: Jun. 3, 2010

(54) METHOD FOR SCREENING IMMUNE MODULATOR

(75) Inventors: Sung-Hoon Kim, Seoul (KR); Jung-Min Han, Seoul (KR)

Correspondence Address: DUANE MORRIS LLP - Atlanta;IP DEPARTMENT, ATLANTIC CENTER PLAZA, 1180 WEST PEACHTREE STREET, NW SUITE 700, ATLANTA, GA, 30309-3348, US

(73) Assignee: Imagene Co., Ltd., Seoul (KR)

(21) Appl. No.: 12/597,874

(22) PCT Filed: Apr. 27, 2007
(86) PCT No.: PCT/KR07/02072

§ 371 Date: Oct. 27, 2009

(51) Int. Cl. G01N 33/53; C12Q 1/68; C07K 16/00

(52) U.S. Cl. 800/3; 435/7.1; 435/6; 530/389.1

(57) ABSTRACT

Disclosed is a method for screening an immune modulator. More specifically, disclosed is a method of screening an immune modulator, an anticancer agent and an agent for treating autoimmune diseases, which regulate the cell surface expression level of gp96, using the binding of the region of amino acids 54-192 of AIMP1 to the region of amino acids 699-799 of AIMP1, set forth in SEQ ID NO: 18. Also disclosed is a method of diagnosing autoimmune diseases using the binding.

### TECHNICAL FIELD

The present invention relates to a method for screening an immune modulator, and more particularly to methods of screening an immune modulator which regulate the cell surface expression level of gp96, an anticancer agent and an agent for treating autoimmune diseases, using the binding of the region of amino acids 54-192 of AIMP1 to the region of amino acids 699-799 of gp96 of SEQ ID NO: 18. Also, the present invention relates to a method of diagnosing autoimmune diseases using the binding.

### BACKGROUND ART

Gp96 is the endoplasmic reticulum (ER)-resident member of the HSP90 family. Gp96 contains a c-terminal KDEL sequence, that is, ER retention signal. However, despite this KDEL sequence, the cell surface expression of gp96 has been demonstrated on mouse Meth-A sarcoma cells, but not on normal embryonic fibroblast cells (Altmeyer A, et. al., Int J Cancer, 69: 340-349, 1996). In addition, it has been reported that HSP species, including gp96, are expressed on murine thymocytes, which indicates that gp96 surface expression is not restricted to tumor cells. Since gp96 has been implicated in innate and adaptive immunity, its cell surface expression may be of immunological relevance. Gp96 has been implicated in the activation or maturation of dendritic cells (DCs). Recently, transgenic mice expressing gp96 on cell surfaces were found to show significant DC activation and spontaneous lupus-like autoimmune disease development (Liu B, et. al., Proc Natl Acad Sci, 100: 15824-15829, 2003). These results suggest that gp96 export from the ER plays an important role of immune regulation, and that the cell surface expression of gp96 must be tightly controlled to avoid unnecessary immune response.

The gp96 was first found to be a tumor rejection antigen having tumor vaccine effects (Srivastava, P. K., et. al., Proc. Natl. Acad. Sci. 83, 3407-3411, 1985) and is currently in a Phase III Clinical Trial for metastatic melanona (Pilla L, et. al., Cancer Immunol Immunother. Aug; 55(8):958-68, 2006). The above-described anticancer effect of the gp96 protein is attributable to the capability to activate immune cells (Arnold-Schild D, et. al., J. Immunol., 1; 162(7):3757-60, 1999) and the capability to act as a kind of chaperone to bind to peptides (Linderoth N A, et. al., J Biol. Chem., 25; 275(8):5472-7, 2000: Singh-Jasuja H, et. al., J Exp Med. 5; 191(11):1965-74, 2000). gp96 binds to cancer cell-specific antigens in cancer cells, and thus has been applied in cancer vaccines (Heikema A, et. al., Immunol Lett. 1; 57(1-3):69-74, 1997). However, it has been found in animal tests that if gp96 is excessively exposed to the surface of normal cells, it induces autoimmune diseases (Liu B, et. al., Proc Natl Acad. Sci., 23; 100(26):15824, 2003). Thus, the regulation of the cell surface expression level of gp96 is important not only in cancer cells, but also in normal cells, and substances regulating the expression level can be developed as anticancer agents or agents for treating autoimmune diseases.

Meanwhile, AIMP1 (ARS-interacting multi-functional protein 1) was previously known as the p43 protein and renamed by the present inventors (Sang Gyu Park et al., Trends in Biochemical Sciences, 30:569-574, 2005). The AIMP1 is a protein consisting of 312 amino acids (Deutscher, M. P., Method Enzymol, 29, 577-583, 1974; Dang C. V. et al., Int. J. Biochem. 14, 539-543, 1982; Mirande, M. et al., EMBO J. 1, 733-736, 1982; Yang D. C. et al., Curr. Top Cell. Regul. 26, 325-335, 1985), which binds to a multi-tRNA synthetase complex to increase the catalytic activity of the multi-tRNA synthetase (Park S. G. et al., J. Biol. Chem. 274, 16673-16676, 1999). AIMP1 is secreted from different types of cells, including prostate cancer, immune and transfected cells. The secretion thereof is induced by various stimuli such as TNFα and heat shock (Park S. G. et al., Am. J. Pathol., 166, 387-398, 2005; Barnett G. et al., Cancer Res. 60, 2850-2857, 2000). It is known that the secreted AIMP1 acts on various target cells, such as monocytes/macrophages, endothelial cells and fibroblasts.

### DISCLOSURE

### Technical Problem

The present inventors have found that the region of amino acids 54-192 of AIMP1, shown in SEQ ID NO: 4, binds directly to the region of amino acids 699-799 of gp96, shown in SEQ ID NO: 18, to regulate the cell surface expression level of gp96, and that if the binding breaks, an autoimmune disease is induced, so that the level of gp96 on the immune cell surface and the serum AIMP1 level in the blood sample of an autoimmune disease patient are higher than those in a normal person, thereby completing the present invention.

It is an object of the present invention to provide an immune modulator, which regulates the cell surface expression level of gp96, and a method for diagnosing autoimmune diseases.

### Technical Solution

To achieve the above objects, in one aspect, the present invention provides a method for screening an immune modulator, the method comprising the steps of:

(a) contacting a test agent with an isolated polypeptide comprising an amino acid sequence set forth in SEQ ID NO: 4; and

(b) testing whether the test agent binds to the isolated polypeptide. The method of the present invention may further comprise the steps of: contacting the test agent, tested in step (b), with an isolated polypeptide comprising an amino acid sequence set forth in SEQ ID NO: 18; and testing whether the test agent binds to the isolated polypeptide comprising the amino acid sequence set forth in SEQ ID NO: 18.

In another aspect, the present invention provides a method for screening an immune modulator, the method comprising the steps of:

(a) contacting a test agent with a cell or tissue expressing an isolated polypeptide, comprising an amino acid sequence set forth in SEQ ID NO: 4, and an isolated polypeptide, comprising an amino acid sequence set forth in SEQ ID NO: 18; and

(b) detecting a change in the cell surface expression level of gp96 in the cell or tissue contacted with the test agent relative to the cell surface expression level of gp96 in a cell or tissue not contacted with the test agent.

In still another aspect, the present invention provides a method for screening an anticancer agent, the method comprising the steps of:

(a) contacting a test agent with an isolated polypeptide comprising an amino acid sequence set forth in SEQ ID NO: 4;

(b) testing whether the test agent binds to the isolated polypeptide;

(c) administering the test agent to a cancer cell or a cancer animal model; and

(d) detecting a change in the progression of cancer in the cancer cell or cancer animal model administered with the test agent.

In still another aspect, the present invention provides a method for screening an anticancer agent, the method comprising the steps of:

(a) contacting a test agent with a cell or tissue expressing a polypeptide comprising an amino acid sequence set forth in SEQ ID NO: 4;

(b) testing whether the cell surface expression level of gp96 in the cell or tissue contacted with the test agent is increased compared to the cell surface expression level of gp96 in a cell not contacted with the test agent;

(c) administering the test agent to a cancer cell or a cancer animal model; and

(d) detecting a change in the progression of cancer in the cancer cell or cancer animal model administered with the test agent.

In yet still another aspect, the present invention provides a method for screening an agent for treating autoimmune diseases, the method comprising the steps of:

(a) contacting a test agent with an isolated polypeptide comprising an amino acid sequence set forth in SEQ ID NO: 18;

(b) testing whether the test agent binds to the isolated polypeptide comprising the amino acid sequence set forth in SEQ ID NO: 18;

(c) administering the test agent to an immune cell or an autoimmune disease animal model; and

(d) measuring the degree of immune suppression in the immune cell or autoimmune disease model administered with the test agent.

In yet another aspect, the present invention provides a method for screening an agent for treating autoimmune diseases, the method comprising the steps of:

(a) contacting a test agent with a cell or tissue expressing an isolated polypeptide comprising an amino acid sequence set forth in SEQ ID NO: 18;

(b) testing whether the cell surface expression level of gp96 in the cell or tissue contacted with the test agent is decreased compared to the cell surface expression level of gp96 in a cell not contacted with the test agent;

(c) administering the test agent to an immune cell or an autoimmune disease animal model; and

(d) measuring the degree of immune suppression in the immune cell or autoimmune disease model administered with the test agent.

In another further aspect, the present invention provides a composition for diagnosing autoimmune diseases, comprising an antibody specific for an AIMP1 protein of SEQ ID NO: 1. In addition, the present invention provides a method for diagnosing autoimmune diseases, the method comprising the steps of: (a) contacting an antibody specific for an AIMP1 protein with a detection sample; (b) forming an antigen-antibody complex; and (c) comparing the amount of formation of the antigen-antibody complex with a control group.

Unless otherwise stated, all technical and scientific terms used herein have the same meanings as commonly understood by those skilled in the art to which the present invention pertains. The following references provide one of skill with a general definition of many of the terms used in the present invention: Singleton et al., DICTIONARY OF MICROBIOLOGY AND MOLECULAR BIOLOTY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY. Also, the following definitions provide aid for the reader in order to execute the present invention. Also, the following definitions are provided to assist the reader in the practice of the invention.

As used herein, the term “expression” means the production of a protein or nucleotide in a cell.

The term “host cell” as used herein refers to a prokaryotic or eukaryotic cell that contains heterologous DNA that has been introduced into the cell by any means, e.g., electroporation, calcium phosphate precipitation, microinjection, transformation, viral infection, etc.

As used herein, the term “isolated” means that the material is removed from its original environment (e.g., the natural environment if it is naturally occurring). For example, it means that a naturally-occurring polynucleotide, polypeptide or cell present in a living animal is not isolated. However, it means that the same polynucleotide, polypeptide or cell separated from some or all of the coexisting materials is isolated, although it is re-inserted in the natural system after it was separated from. Such polynucleotide can be part of a vector and/or such polynucleotide or polypeptide can be part of a composition. Such vector or composition is not part of its natural environment, but isolated.

As used herein, the term “immune modulator” refers to an agent which increases or decreases the cell surface expression level of gp96 to enhance or suppress immunity.

As used herein, the term “regulating the cell surface expression level of gp96” may be the up-regulation (i.e., activation or stimulation) or down-regulation (i.e., suppression or inhibition) of cell surface expression level of gp96. For example, when the cell surface expression level of gp96 is down-regulated, AIMP1 binds to the gp96 protein to inhibit the migration of gp96 to the cell surface, thus suppressing an immune response, and when the cell surface expression level of gp96 is up-regulated, AIMP1 is deleted to increase the migration of the gp96 to the cell surface, thus increasing an immune response.

As used herein, the term “polypeptide” is used interchangeably with the terms “polypeptides” and “protein(s)”, and refers to a polymer of amino acid residues, e.g., as typically found in proteins in nature.

As used herein, the term “isolated polypeptide” refers to either a polypeptide comprising an amino acid sequence of SEQ ID NO: 4 or a polypeptide comprising an amino acid sequence of SEQ ID NO: 18. The polypeptide having the amino acid sequence of SEQ ID NO: 4 is a polypeptide having part of the amino acid sequence of the AIMP1 protein, that is, the region of amino acids 54-192 of SEQ ID NO: 1, and the polypeptide having the amino acid sequence of SEQ ID NO: 18 refers to a polypeptide having part of the C-terminal amino acid sequence of the gp96 protein, that is, the region of amino acids 699-799 of SEQ ID NO: 13.

Also, the scope of the inventive polypeptide includes functional equivalents of the polypeptide having the amino acid sequence of SEQ ID NO: 4, and salts thereof, and functional equivalents of the polypeptide having the amino acid sequence of SEQ ID NO: 18, and salts thereof.

As used herein, the term “sequence identity or homology” is defined herein as the percentage of amino acid residues in the candidate sequence that are identical with amino acid sequence of SEQ ID NO: 4 or SEQ ID NO: 18, after aligning the sequences and introducing gaps. If necessary, to achieve the maximum percent sequence identity, any conservative substitutions are not considered as part of the sequence identity. None of N-terminal, C-terminal, or internal extensions, deletions, or insertions into the amino acid sequence of SEQ ID NO: 4 or SEQ ID NO: 18 shall be construed as affecting sequence identity or homology. Thus, sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a predetermined portion of one or both sequences). The programs provide a default opening penalty and a default gap penalty, and a scoring matrix, such as PAM 250 (a standard scoring matrix; see Dayhoff et al., in Atlas of Protein Sequence and Structure, vol. 5, supp. 3 (1978)), can be used in conjunction with the computer program. For example, the percent identity can be calculated as: the total number of identical matches multiplied by 100 and then divided by the sum of the length of the longer sequence within the matched span and the number of gaps introduced into the longer sequences in order to align the two sequences.

The polypeptide according to the present invention may be extracted from the nature or constructed by a genetic engineering method. For example, a DNA sequence (e.g., SEQ ID NO: 5) encoding the amino acid sequence of SEQ ID NO: 4 or a functional equivalent thereof is constructed according to any conventional method. Also, a DNA sequence (e.g., SEQ ID NO: 19) encoding the amino acid sequence of SEQ ID NO: 18 or a functional equivalent thereof is constructed according to any conventional method. The DNA sequence may synthesized by performing PCR using suitable primers (e.g., SEQ ID NOS: 26 and 27). Alternatively, the DNA sequence may also be synthesized by a standard method known in the art, for example using an automatic DNA synthesizer (commercially available from Biosearch or Applied Biosystems). The constructed DNA sequence is inserted into a vector comprising at least one expression control sequence that is operatively linked to the DNA sequence so as to control the expression of the DNA molecule, and host cells are transformed with the resulting recombinant expression vector. The transformed cells are cultured in a medium and condition suitable to express the DNA sequence, and a substantially pure polypeptide encoded by the DNA sequence is collected from the culture medium. The collection of the pure polypeptide may be performed using a method known in the art, for example, chromatography. In this regard, the term “substantially pure polypeptide” means the inventive polypeptide that does not substantially contain any other proteins derived from host cells. For the genetic engineering method for synthesizing the inventive polypeptide, the reader may refer to the following literatures: Maniatis et al., Molecular Cloning: A Laboratory Manual, Cold Spring Harbor Laboratory 1982; Sambrook et al., supra; Gene Expression Technology, Method in Enzymology, Genetics and Molecular Biology, Method in Enzymology, Guthrie & Fink (eds.), Academic Press, San Diego, Calif. 1991; and Hitzeman et al., J. Biol. Chem., 255, 12073-12080 1990.

Alternatively, the inventive peptide can be chemically synthesized according to any technique known in the art (Creighton, Proteins: Structures and Molecular Principles, W.H. Freeman and Co., NY 1983). For example, the inventive peptide can be prepared by conventional liquid or solid phase synthesis, fragment condensation, F-MOC or T-BOC chemistry (Chemical Approaches to the Synthesis of Peptides and Proteins, Williams et al., Eds., CRC Press, Boca Raton Fla., 1997; A Practical Approach, Atherton & Sheppard, Eds., IRL Press, Oxford, England, 1989).

As used herein, the term “nucleic acid”, “DNA sequence” or “polynucleotide” refers to a deoxyribonucleotide or ribonucleotide polymer in either single- or double stranded form. Unless otherwise limited, it encompasses known analogues of natural nucleotides that hybridize to nucleic acids in a manner similar to naturally occurring nucleotides.

As used herein, the term “nucleic acid sequence” includes all DNA, cDNA and RNA sequences. Specifically, the polynucleotide may have either a base sequence encoding the amino acid sequence of SEQ ID NO: 4 or a base sequence complementary thereto. Preferably, it may have a base sequence set forth in SEQ ID NO: 5 or SEQ ID NO: 19. The nucleic acid may be isolated from the nature or may be constructed by a genetic engineering method as described above.

The term “analog” as used herein refers to a molecule that structurally resembles a reference molecule, but that has been modified in a target and controlled manner, by replacing a specific substituent of the reference molecule with an alternate substituent. Compared to the reference molecule, an analog would be expected, by one skilled in the art, to exhibit the same, similar or improved utility. Synthesis and screening of analogs, in order to identify variants of known compounds having improved traits (such as higher binding affinity for a target molecule) is an approach that is well known in pharmaceutical chemistry.

As used herein, the term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence.

As used herein, the term “contacting” has its normal meaning and refers to combining two or more agents (e.g., polypeptides) or combining agents and cells (e.g., a protein and a cell). Contacting can occur in vitro, e.g., combining two or more agents or combining a test agent, and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

As used herein, the term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

More specifically, test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purines, pyrimidines, or their derivatives, structural analogs or combinations thereof. Some test agents are synthetic molecules, and others are natural molecules. The test agents can be obtained from a wide variety of sources including libraries of synthetic or natural compounds. Combinatorial libraries can be produced for many types of compounds that can be synthesized in a step-by-step fashion. Large combinatorial libraries of compounds can be constructed by the encoded synthetic libraries (ESL) method (WO 95/12608, WO 93/06121, WO 94/08051, WO 95/35503 and WO 95/30642). Peptide libraries can also be generated by phage display methods (WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, and amidification, to produce structural analogs.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

Also, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of p53. A number of assays are available for such screening (Shultz, Bioorg. Med. Chem. Lett., 8:2409-2414, 1998; Weller, Mol. Drivers., 3:61-70, 1997; Fernandes, Curr. Opin. Chem. Biol., 2:597-603, 1998; and Sittampalam, Curr. Opin. Chem. Biol., 1:384-91, 1997).

Libraries of test agents to be screened according to the method of the present invention can also be generated based on structural studies of AIMP1, their fragments or analogs and on structural studies of gp96, their fragments or analogs. Such structural studies allow the identification of test agents that are more likely to bind to AIMP1 or gp96. The three-dimensional structure of AIMP1 or gp96 can be studied in a number of ways, e.g., crystal structure and molecular modeling. Methods of studying protein structures using x-ray crystallography are well known in the literature: Physical Bio-Chemistry, Van Holde, K. E. (Prentice-Hall, New Jersey 1971), pp. 221-239, and Physical Chemistry with Applications to the Life Sciences, D. Eisengerg & D.C. Crothers (Benjamin Cummings, Menlo Park 1979).

Computer modeling of AIMP1 structure provides another means for designing test agents for screening immune modulators regulating the cell surface expression level of gp96. Methods of molecular modeling have been described in the literature: U.S. Pat. No. 5,612,894 and U.S. Pat. No. 5,583,973. Also, protein structures can be determined by neutron diffraction and NMR (nuclear magnetic resonance): Physical Chemistry, 4^{th} Ed. Moore, W. J. (Prentice-Hall, New Jersey 1972) and NMR of Proteins and Nucleic Acids, K. Wuthrich (Wiley-Interscience, New York 1986).

The term “antibody” as used herein means a specific protein molecule that indicates an antigenic region. With respect to the objects of the present invention, the antibody refers to an antibody specifically recognizing AIMP1 and includes all polyclonal and monoclonal antibodies. Antibodies against the AIMP1 protein may be easily prepared in accordance with conventional technologies known to one skilled in the art. The AIMP1 of the present invention may have the amino acid sequence set forth in SEQ ID NO: 1.

Polyclonal antibodies may be prepared by a method widely known in the art, which includes injecting the AIMP1 protein into an animal and collecting blood samples from the animal to obtain serum containing antibodies. Such polyclonal antibodies may be prepared from a certain animal host, such as goats, rabbits, sheep, monkeys, horses, pigs, cows and dogs.

Monoclonal antibodies may be prepared by a method widely known in the art, such as a fusion method (Kohler and Milstein, European Journal of Immunology, 6:511-519 (1976)), a recombinant DNA method (U.S. Pat. No. 4,816,567) or a phage antibody library technique (Clackson et al, Nature, 352:624-628 (1991); and Marks et al, J. Mol. Biol., 222:58, 1-597 (1991)).

Also, the antibodies that are used to detect the AIMP1 protein include complete forms having two full-length light chains and two full-length heavy chains, as well as functional fragments of antibody molecules. The functional fragments of antibody molecules refer to fragments retaining at least an antigen-binding function, and include Fab, F (ab′), F (ab′)₂, Fv and the like.

As used herein, the term “detecting sample” means a biological sample, such as tissues, cells, whole blood, serum, plasma, saliva, semen, cerebrospinal fluid or urine, that can detect the difference in amount of expressed marker proteins caused by the autoimmune diseases induction, and the sample is prepared through the treatment according to the methods widely known in the art.

As used herein, the term “antigen-antibody complex” means a complex of the AIMP1 protein in a sample with an antibody that specifically recognizes the AIMP1 protein.

An experimental method used to confirm the formation of the autoantibody-antigen complex includes, but is not limited to, Immunohistological staining, Radioimmunoassay (RIA), Enzyme-Linked Immunosorbent Assay (ELISA), Western Blotting, Immunoprecipitation Assay, Immunodiffusion Assay, Complement Fixation Assay, FACS, protein chip, etc.

Hereinafter, the present invention will be described in detail.

The present inventors found through a binding affinity test that AIMP1 was bound to gp96 (see FIG. 1). Also, the present inventors performed Western blot analysis and co-immunoprecipitation and, as a result, it was confirmed again that gp96 was bound directly to AIMP1 (see FIGS. 2 to 4). In order to examine the intracellular location of gp96 by binding to AIMP1, MEF cells were isolated from each of AIMP1 wild-type mice (AIMP1^{+/+})⁻ and AIMP1-deleted mice (AIMP1^{-/-}), and the locations of gp96 in the isolated cells were examined. As a result, in the AIMP1 wild-type mice, gp96 was found mainly in endoplasmic reticulum (ER) around the cell nucleus, but in the AIMP1-deleted mice, gp96 was found in the plasma membrane (see FIG. 5). When AIMP1 was overexpressed in the AIMP1-deleted mice, it was shown that gp96 was also found in ER (see FIG. 6). These results suggest that the intracellular location of gp96 is regulated by AMP1.

Moreover, the cell surface expression level of gp96 by AIMP1 was analyzed by FACS, and as a result, the cell surface expression levels of gp96 in the MEF cells and spleen cells of the AMP1-deleted mice (AIMP1^{-/-}) were increased compared to the cell surface expression levels of gp96 in the MEF cells and spleen cells of the wild-type mice (AIMP1^{+/+}) (see FIGS. 7 to 9). When HeLa cells were treated with AIMP1 siRNA to intrinsically inhibit AIMP1, the cell surface expression level of gp96 was increased (see FIG. 10), and when AIMP1 was overexpressed in 293 cells, the cell surface expression level of gp96 was decreased (see FIG. 11). This suggests that the cell surface expression level of gp96 is regulated by AIMP1. This can further be confirmed by the fact that an autoimmune phenotype appeared in the AIMP1-deleted mice (see FIGS. 12 to 14). Specifically, it could be seen that the cell surface expression level of gp96 was regulated by AIMP1, and when AIMP1 was deleted, the cell surface expression level of gp96 was increased, so that a strong immune response occurred, thus causing autoimmune diseases.

As described above, it was found that AIMP1 was bound to gp96 and that the cell surface expression level of gp96 was regulated by AIMP1. The present inventors identified the binding regions of AIMP1 and gp96. As a result, the region of amino acids 54-192 (SEQ ID NO: 4) of AIMP1 having an amino acid sequence of SEQ ID NO: 1 was bound to the region of amino acids 699-799 (SEQ ID NO: 18) of gp96 having an amino acid sequence of SEQ ID NO: 13 (see FIG. 18).

In summary, the region of amino acids 54-192 of AIMP1 as set forth in SEQ ID NO: 4 binds directly to the region of amino acids 699-799 of gp96 to assist the endoplasmic reticulum (ER) retention of gp96 to inhibit the migration of gp96 to the cell surface. On the other hand, when AIMP1 is deleted, the migration of gp96 to the cell surface increases to induce an increase in immune response. Thus, a substance capable of attenuating or enhancing the binding between the fragments can be developed as an anticancer vaccine or an immunosuppressant agent. A system of screening an immune modulator using the binding between the region of amino acids of 54-192 of AIMP1 as set forth in SEQ ID NO: 4 and the region of amino acids 699-799 of gp96 as set forth in SEQ ID NO: 18 was disclosed for the first time in the present invention.

As described above, AIMP1 binds to gp96 to regulate the intracellular location of gp96 and, as a result, the amount of gp96 on the cell surface and the resulting immune response are regulated. It has been found in animal tests that, if gp96 is excessively exposed to the surface of normal cells, it induces autoimmune diseases (Liu B, et. al., Proc Natl Acad Sci, 100: 15824-15829, 2003). It was seen that, for autoimmune patients, the binding between gp96 and AIMP1 in cells was broken, so that gp96 was highly expressed on the cell surface, and AIMP1 was secreted out of the cells and present in blood at a high level (see FIG. 19). Specifically, it could be seen that the level of AIMP1 in the sera of SLE patients was higher than the level of AIMP1 in the sera of normal persons (see FIG. 19). This suggests that an antibody to AIMP1, which allows the blood level of AIMP1 to be measured, can be used as a novel marker capable of diagnosing autoimmune diseases.

Accordingly, the present invention provides a method for screening an immune modulator, comprising the steps of: (a) contacting a test agent with an isolated polypeptide comprising an amino acid sequence set forth in SEQ ID NO: 4; and (b) testing whether the test agent binds to the isolated polypeptide.

In another aspect, the present invention provides a method for screening an immune modulator, further comprising the steps of: contacting the test agent, tested in the step (b), with an isolated polypeptide comprising an amino acid sequence set forth in SEQ ID NO: 18; and testing whether the candidate substance binds to the isolated polypeptide comprising the amino acid sequence set forth in SEQ ID NO: 18.

Various biochemical and molecular biological techniques known in the art can be employed to perform the above methods. Such techniques are described in: Sambrook et al., Molecular Cloning: A Laboratory Manual, Cold Spring Harbor Press, N.Y., Second (1998) and Third (2000) Editions; and Ausubel et al., Current Protocols in Molecular Biology, John Wiley & Sons, Inc., New York (1987-1999).

In order to screen an immune modulator according to the present invention, whether the isolated polypeptide comprising the region of amino acids 54-192 (SEQ ID NO: 4) of AIMP1 having an amino acid sequence of SEQ ID NO: 1 contacts with a test agent can be determined by contacting the test agent with the isolated polypeptide. The contacting of the test agent with the isolated polypeptide can be assayed by a number of methods including, e.g., labeled in vitro protein-protein binding assays, electrophoretic mobility shift assays (EMSA), immunoassays for protein binding, functional assays (phosphorylation assays, etc.), and the like (U.S. Pat. Nos. 4,366,241: 4,376,110; 4,517,288 and 4,837,168; and Bevan et al., Trends in Biotechnology, 13:115-122, 1995; Ecker et al., Bio/Technology, 13:351-360, 1995; and Hodgson, Bio/Technology, 10:973-980, 1992). The test agent can be identified by detecting a direct binding to the isolated polypeptide comprising the amino acid sequence set forth in SEQ ID NO: 4. For example, the test agent can be identified by detecting co-immunoprecipitation with the AIMP1 polypeptide using an antibody directed to the AIMP1 protein comprising the amino acid sequence set forth in SEQ ID NO: 4. The test agent can also be identified by detecting a signal that indicates that the agent binds to the isolated polypeptide or AIMP1, e.g., fluorescence quenching.

Competition assays provide a suitable format for identifying a test agent that specifically binds to the isolated polypeptide or AIMP1 of the present invention. In such formats, a test agent is screened in competition with a compound already known to bind to AIMP1. The known binding compound can be a synthetic compound. It can also be an antibody, which specifically recognizes the AIMP1, e.g., a monoclonal antibody directed against the PDX1 polypeptide. If the test agent inhibits binding of the compound known to bind the isolated polypeptide or AIMP1, then the test agent also binds the isolated polypeptide or AIMP1 of the present invention.

Numerous types of competitive binding assays are known. Examples thereof include solid phase direct or indirect radioimmunoassay (RIA), solid phase direct or indirect enzyme immunoassay (EIA), sandwich competition assay (Stahli et al., Methods in Enzymology 9:242 253 (1983)); solid phase direct biotin-avidin EIA (Kirkland et al., J. Immunol. 137:3614 3619 (1986)); solid phase direct labeled assay, solid phase direct labeled sandwich assay (Harlow and Lane, “Antibodies, A Laboratory Manual,” Cold Spring Harbor Press (1988)); solid phase direct label RIA using ¹²⁵I label (Morel et al., Mol. Immunol. 25(1):7 15 (1988)); solid phase direct biotin-avidin EIA (Cheung et al., Virology 176:546 552 (1990)); and direct labeled RIA (Moldenhauer et al., Scand. J. Immunol. 32:77 82 (1990)). Typically, such assays involve the use of purified polypeptide bound to a solid surface or cells bearing either of these, an unlabelled test agent and a labeled reference compound. Competitive inhibition is measured by determining the amount of label bound to the solid surface or cells in the presence of the test agent. Test agents identified by competition assay include agent binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize the isolated polypeptide or AIMP1 of the present invention or its fragments onto a solid phase matrix. The solid phase matrix is then put in contact with a test agent, for an interval sufficient to allow the test agent to bind. After washing away any unbound material from the solid phase matrix, the presence of the agent bound to the solid phase was confirmed. The methods can further include the step of separating the agent by eluting the bound agent from the solid phase matrix, thereby isolating the agent. Alternatively, other than immobilizing the isolated polypeptide or AIMP1 of the present invention, the test agent is bound to the solid matrix, and the isolated polypeptide or AIMP1 of the present invention is then added.

Soluble assays include some of the combinatory libraries screening methods described above. Under the soluble assay formats, neither the test agent nor the isolated polypeptide or AIMP1 of the present invention is bound to a solid support. Binding of the isolated polypeptide or AIMP1 of the present invention to a test agent can be determined by, for exmaple, changes in fluorescence of either the isolated polypeptide or AIMP1 of the present invention and/or the test agent. Fluorescence may be intrinsic or conferred by labeling of component with a fluorophor.

In some binding assays, either the isolated polypeptide or AIMP1 of the present invention, the test agent or a third molecule (e.g., an antibody binding to AIMP1) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵1, ³²P, ³⁵S) or a chemiluminescent or fluorescent group. Similarly, the detectable group can be a substrate, cofactor, inhibitor or affinity ligand.

Because the isolated polypeptide binds to gp96 to regulate the cell surface expression level of gp96, a test agent binding to the isolated polypeptide comprising the amino acid sequence set forth in SEQ ID NO: 4 may be used as an immune modulator capable of increasing the cell surface expression level of gp96 to enhance immunity.

If the test agent does not bind to the isolated polypeptide comprising the amino acid sequence set forth in SEQ ID NO: 4, the test agent can be brought into contact with the isolated polypeptide comprising the amino acid sequence set forth in SEQ ID NO: 18 to test whether the test agent binds to the isolated polypeptide comprising the amino acid sequence set forth in SEQ ID NO: 18. If the test agent binds to the isolated polypeptide, it may be used as an immune modulator capable of inhibiting immunity by decreasing the cell surface expression level of the gp96 protein comprising the amino acid sequence set forth in SEQ ID NO: 18.

Binding of the test agent to the isolated polypeptide can be measured in the same manner as described above.

The screening method of the present invention may comprise the steps of: contacting a test agent with a cell or tissue expressing the isolated polypeptide, comprising the amino acid sequence set forth in SEQ ID NO: 4, and the isolated polypeptide, comprising the amino acid sequence set forth in SEQ ID NO: 18; and (b) detecting a change in the cell surface expression level of gp96 in the cell or tissue contacted with the test agent relative to the cell surface expression level of gp96 in a cell or tissue not contacted with the test agent.

The cell may be a cell in which the polypeptides are intrinsically expressed. Alternatively, it may also be a recombinant cell obtained by transfecting the cell simultaneously with an isolated polynucleotide comprising a nucleic acid sequence encoding the amino acid sequence of SEQ ID NO: 4 and with an isolated polynucleotide comprising a nucleic acid sequence encoding the amino acid sequence of SEQ ID NO: 18.

The cell surface expression level of gp96 can be measured according to any method known in the art. For example, the cell surface expression level of gp96 can be measured by labeling antibody to gp96 with a label such as immunofluorescent label, and observing the labeled antibody with a microscope or performing FACS analysis.

The region of amino acids 54-192 of AIMP1 binds directly to the region of amino acids 699-799 of gp96 to assist the ER retention of gp96 so as to inhibit the migration of gp96 to the cell surface, thus suppressing immune responses. Accordingly, a test agent regulating the interaction between the isolated polypeptide, comprising the amino acid sequence of SEQ ID NO: 4, that is, the region of amino acids 54-192 of AIMP1, and the isolated polypeptide, comprising the amino acid sequence of SEQ ID NO: 18, that is, the region of amino acids 699-799 of gp96, can be used as an immune modulator that regulates the cell surface expression level of gp96. The test agent can be used as an immune modulator that stimulates or enhances the interaction between the polypeptides to inhibit immunity. On the contrary, the test agent can be used as an immune modulator that inhibits or attenuates the interaction between the polypeptides to increase immunity.

The screening method can be performed using various methods known in the art, including labeled in vitro protein-protein binding assays (in vitro pull-down assays), electrophoretic mobility shift assays (EMSA), immunoassays for protein binding, functional assays (phosphorylation assays, etc.), yeast-2 hybrid assays, immunoprecipitation assays, immunoprecipitation Western blot assays, immuno-co-localization, and the like.

For example, yeast-2 hybrid assays can be performed using yeasts expressing a partial fragment polypeptide of AIMP1, comprising the amino acid sequence of SEQ ID NO: 4, and/or AIMP1, and a partial fragment polypeptide of gp96, comprising the amino acid sequence of SEQ ID NO: 18, and/or gp96, or parts or homologues of these proteins, fused respectively to the bacterial repressor LexA or to the DNA-binding domain of yeast GAL4 and to the transactivation domain of the yeast GAL4 protein (KIM, M. J. et al., Nat. Gent., 34:330-336, 2003). Interaction of the partial fragment of AIMP1, comprising the amino acid sequence of SEQ ID NO: 4, and/or AIMP1, with the partial fragment of gp96, comprising the amino acid sequence of SEQ ID NO: 18, and/or gp96, makes it possible to reconstitute a transactivator which induces expression of a reporter gene placed under the control of a promoter having a regulatory sequence to which attaches the LexA protein or the DNA-binding domain of GAL4.

As the reporter gene, a known gene encoding any detectable polypeptide, such as CAT (chloramphenicol acetyltransferase), luciferase, beta-galactosidase, beta-glucosidase, alkaline phosphatase or GFP (green fluorescent protein), may be used. If the interaction between AIMP1 and gp96, or parts or homologues of these proteins, is stimulated or enhanced by the test agent, the expression of the reporter gene will be increased compared to that in normal conditions. On the contrary, if the interaction is suppressed or attenuated by the test agent, the reporter gene will not be expressed or will be less expressed compared to that in normal conditions.

Also, a reporter gene will be chosen which encodes a protein which allows growth of yeast under conditions where this growth is inhibited when there is no expression of said reporter gene. This reporter gene will, for example, be an auxotrophic gene encoding an enzyme involved in a biosynthetic pathway for amino acids or nitrogenous bases, such as the yeast genes ADE3, HIS3, etc., or equivalent genes originating from other organisms. When the interaction between AIMP1 and gp96, or parts or homologues of these proteins, expressed in this system, is inhibited or attenuated by the test agent, the reporter gene will not be expressed or will be less well expressed, thus inducing arrest or slowing down of yeast growth under the above conditions. This effect of expression of this reporter gene may be visible to the naked eye or via devices (e.g., microscopes).

In another aspect, the present invention provides a method for screening an anticancer agent, the method comprising the steps of:

(a) contacting a test agent with an isolated polypeptide comprising an amino acid sequence set forth in SEQ ID NO: 4;

(b) testing whether the test agent binds to the isolated polypeptide;

(c) administering the test agent to a cancer cell or a cancer animal model; and

(d) detecting a change in the progression of cancer in the cancer cell or cancer animal model administered with the test agent.

In still another aspect, the present invention provides a method for screening an anticancer agent, the method comprising the steps of:

(a) contacting a test agent with a cell or tissue into contact with a cell or tissue expressing an isolated polypeptide comprising an amino acid sequence set forth in SEQ ID NO: 4;

(b) testing whether the cell surface expression level of gp96 in the cell or tissue contacted with the test agent is increased compared to the cell surface expression level of gp96 in a cell not contacted with the test agent;

(c) administering the test agent to a cancer cell or a cancer animal model; and

(d) detecting a change in the progression of the cancer cell or cancer animal model administered with the test agent.

As described above, if the test agent binds to the isolated polypeptide comprising the amino acid sequence of SEQ ID NO: 4 or increases the cell surface expression level of gp96 in the cell expressing the polypeptide, it can be used as an immune modulator that causes gp96 to migrate to the cell surface to induce an increase in immune response. If the test agent is administered to a cancer cell or a cancer animal model and confirmed to inhibit the progression of cancer, it can be used as a novel anticancer agent. A gp96 cancer vaccine, which is in a Phase III Clinical Trial, is problematic in quantity and cost because it must be obtained from a cancer patient. However, the anticancer agent screened according to the method of the present invention can be developed as an anticancer agent which can substitute for the gp96 cancer vaccine. The cancer cell or cancer animal model can be obtained from depository institutions, be commercially available or be constructed according to any method known in the art.

Examples of the cancer may include, but are not limited to, melanoma, breast cancer, rectal cancer, lung cancer, small-cell lung cancer, stomach cancer, liver cancer, blood cancer, bone cancer, pancreatic cancer, skin cancer, head or neck cancer, skin or intraocular melanoma, uterine carcinoma, ovarian cancer, colorectal cancer, cancer near the anus, colon cancer, oviduct carcinoma, endometrial carcinoma, cervical cancer, vaginal cancer, vulva carcinoma, Hodgkin's disease, esophagus cancer, small intestinal tumor, endocrine gland cancer, thyroid cancer, parathyroid cancer, adrenal cancer, soft-tissue sarcoma, uterine cancer, penis cancer, prostate cancer, chronic or acute leukemia, lymphocytic lymphoma, bladder cancer, kidney or urethra cancer, kidney cell carcinoma, kidney pelvis carcinoma, CNS tumor, primary CNS lymphoma, spinal tumor, brain stem glioma, and pituitary adenoma, and a combination of one or more thereof. Preferably, the cancer is melanoma.

In still another aspect, the present invention provides a method for screening an agent for treating autoimmune diseases, the method comprising the steps of:

(a) contacting a test agent with an isolated polypeptide comprising an amino acid sequence set forth in SEQ ID NO: 18;

(d) testing whether the test agent binds to the isolated polypeptide comprising the amino acid sequence of SE ID NO: 18;

(e) administering the test agent to an immune cell or an autoimmune disease animal model; and

(f) measuring the degree of immune suppression in the immune cell or autoimmune disease animal model administered with the test agent.

In still another aspect, the present invention provides a method for screening an agent for treating autoimmune diseases, the method comprising the steps of:

(a) contacting a test agent with a cell or tissue expressing an isolated polypeptide comprising an amino acid sequence set forth in SEQ ID NO: 18;

(b) testing whether the cell surface expression level of gp96 in the cell or tissue contacted with the test agent is increased compared to the cell surface expression level of gp96 in a cell not contact with the test agent;

(c) administering the test agent to an immune cell or an autoimmune disease animal model; and

(d) measuring the degree of immune suppression in the immune cell or autoimmune disease animal model administered with the test agent.

In yet another aspect, the present invention provides a method for screening an agent for treating autoimmune diseases, the method comprising the steps of:

(a) contacting a candidate substance either with an isolated polypeptide comprising an amino acid sequence set forth in SEQ ID NO: 4 or with a cell or tissue expressing the polypeptide;

(b) testing whether the candidate substance binds to the isolated polypeptide or to the cell or tissue expressing the isolated polypeptide;

(c) contacting the candidate substance, tested in the step (b),

either with an isolated polypeptide comprising an amino acid sequence set forth in SEQ ID NO: 18 or with a cell or tissue expressing the polypeptide of SEQ ID NO: 18;

(d) testing whether the candidate substance binds to the isolated polypeptide comprising the amino acid sequence of SEQ ID NO: 18 or to the cell or tissue expressing the polypeptide of SEQ ID NO: 18;

(e) administering the candidate substance to an immune cell or an autoimmune disease animal model; and

(f) measuring the degree of immune suppression in the immune cell or autoimmune disease animal model administered with the candidate substance.

As described above, if the test agent binds to the isolated polypeptide comprising the amino acid sequence set forth in SEQ ID NO: 18 or decreases the cell surface expression level of gp96 in the cell expressing the polypeptide, it can be used as an immune modulator that inhibits the migration of gp96 to the cell surface to inhibit immune responses. If the test agent is administered to the immune cell or autoimmune disease animal model and confirmed to suppress immunity in the cell or animal model, it can be used as a novel agent for treating autoimmune diseases.

The immune cell or autoimmune disease model can be obtained from depository institutions, be commercially available or be constructed according to any method known in the art. Examples of the immune cell include, but are not limited to, dendritic cells, T cell, B cells, macrophage cells and the like, and examples of the autoimmune disease animal model include, but art not limited to, AIMP1-deleted mice (Cecconi, F. & Meyer, B. I., FEBS Lett., 480:63-71, 2000), and transgenic mice expressing gp96 on the cell surface (Liu B, et. al., Proc Natl. Acad. Sci. USA, 100:15824-15829, 2003). Examples of the autoimmune diseases include systemic lupus erythematosus, rheumatoid arthritis, multiple sclerosis, diabetes, Hashimoto's thyroiditis, psoriasis, scleroderma, inflammatory bowel disease and myasthenia gravis.

In yet another aspect, the present invention provides a composition for diagnosing autoimmune diseases comprising an AIMP1-specific antibody, whether the AIMP1-specific antibody is capable of measuring the level of the AIMP1 protein.

The inventive composition for diagnosing autoimmune diseases comprises an antibody specifically recognizing the AIMP1 protein, and tools and reagents, which are generally used for immunological assays in the art as well. Such tools/reagents include, but are not limited to, suitable carriers, labeling substances capable of generating detectable signals, solubilizing agents, detergents, buffering agents, and stabilizing agents. When the labeling substance is an enzyme, the composition may include a substrate allowing the measurement of enzyme activity and a reaction terminator. Suitable carriers include, but are not limited to, soluble carriers, for example, physiologically acceptable buffers known in the art, for example, PBS, insoluble carriers, for example polymers such as polystylene, polyethylene, polypropylene, polyester, polyacrylnitrile, fluorocarbon resin, crosslinked dextran, polysaccharides and magnetic microparticles composed of latex plated with metals, papers, glasses, metals, agarose, and combinations thereof.

The inventive composition for diagnosing autoimmune diseases may be in the form of, but is not limited to, dipstick-type devices, immunochromatographic test strips and radial partition immunoassay devices, and flow-through devices.

In yet another aspect, the present invention provides a method for diagnosing autoimmune diseases, which comprises the steps of: contacting an AIMP1-specific antibody with a detection sample; and comparing the formation of an antigen-antibody complex in the sample with that in a control group.

Labels allowing qualitative or quantitative analysis of the formation of the antigen-antibody complex include, but are not limited to, enzymes, fluorophores, ligands, luminophores, microparticles, redox molecules and radioisotopes. The enzymes that can be used as the detection levels include, but not are limited to, β-glucuronidase, β-D-glucosidase, β-D-galactosidase, urease, peroxidase, alkaline phosphatase, acetylcholinesterase, glucose oxidase, hexokinase, GDPase, RNase, glucose oxidase, luciferase, phosphofructokinase, phosphoenolpyruvate carboxylase, aspartate aminotransferase, phosphenolpyruvate decarboxylase, β-lactamase. The fluorophores include, but are not limited to, fluorescein, isothiocyanate, rhodamine, phycoerythrin, phycocyanin, allophysocyanin, o-phthalate and fluorescamine. The ligands include, but are not limited to, biotin derivatives. The luminophores include, but are not limited to, acridinium ester, luciferin and luciferase. The microparticles include, but are not limited to, colloidal gold and colored latex. The redox molecules include, but are not limited to, ferrocene, lutenium complex compound, viologen, quinone, Ti ion, Cs ion, diimide, 1,4-benzoquinone, hydroquinone, K₄ W(CN)₈, [Os(bpy)₃]²⁺, [Ru(bpy)₃]²⁺ and [Mo(CN)₈]⁴−. The radioisotopes include, but are not limited to, ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I and ¹³¹I ¹⁸⁶Re.

Whether there is a significant difference in the formation of antigen-antibody complexes between the control group and the detection sample can be examined through an absolute (e.g., μg/me) or relative (e.g., relative signal intensity), thus diagnosing an autoimmune disease.

### DESCRIPTION OF DRAWINGS

FIG. 1 shows the results of silver staining of a protein, isolated from mouse pancreas and purified with biotin-conjugated AIMP1. In FIG. 1, the arrows indicate AIMP1-bound proteins.

FIG. 2 shows the results of Western blot analysis of a protein, isolated from mouse pancreas and purified with biotin-conjugated AIMP1.

FIG. 3 shows the results of Western blot analysis of proteins, isolated from the pancreases of AIMP1-deleted mice (−/−) and wild-type mice (+/+) and purified with GST-AIMP1.

FIG. 4 shows the results of co-immunoprecipitation with anti-gp96 antibody for proteins isolated from HeLa cells.

FIG. 5 shows the results of immunofluorescent staining conducted to examine the intracellular location of gp96 in MEFs, derived from AIMP1-deleted mice (−/−) and wild-type mice (+/+). In FIG. 5, ER: endoplasmic reticulum; and PM: plasma membrane.

FIG. 6 shows the results of immunofluorecent staining conducted to examine the intracellular location of gp96 in AIMP1-deleted mouse (−/−)-derived MEFs, transformed with myc-AIMP1.

FIG. 7 shows the results of FACS analysis of stained gp96 on the cell surface in MEFs, derived from wild-type mice (+/+) and AIMP1-deleted mice (−/−).

FIG. 8 shows the results of FACS analysis of stained gp96 on the cell surface in splenocytes, derived from wild-type mice (+/+) and AIMP1-deleted mice (−/−).

FIG. 9 shows the results of immunofluorescent staining with anti-gp96 antibody (green) or anti-Fas antibody (green) in splenocytes, derived from wild-type mice (+/+) and AIMP1-deleted mice (−/−). In FIG. 9, the nuclei were stained with PI (red).

FIG. 10 shows the results of FACS analysis (left) and Western blot analysis (right), conducted to analyze the cell surface expression level of gp96 in HeLa cells, treated with a control group or AIMP1 siRNA.

FIG. 11 shows the results of FACS analysis (left) and Western blot analysis (right), conducted to analyze the cell surface expression level of gp96 in 293 cells, transfected with an empty vector (EV) or an AIMP1 vector.

FIG. 12 shows the results of Western blot analysis, conducted with autologous serum to analyze nuclear proteins isolated from the livers of wild-type mice (+/+) and AIMP1-deleted mice (−/−).

FIG. 13 shows the results of immunofluorescent staining, conducted to examine whether antinuclear antibody (ANA) is present in the sera of wild-type mice (+/+) and AIMP1-deleted mice (−/−) (upper portion), and shows the results of observation for the deposition of immune complexes in glomeruli (lower portion).

FIG. 14 shows the serum Ig levels of wild-type mice (+/+), AIMP1 heterozygous mice (+/−) and AIMP1-deleted mice (−/−).

FIG. 15 shows the Western blot analysis conducted to analyze the binding of gp96 to purified GST-AIMP1 fragments.

FIG. 16 shows the results of Western blot analysis conducted to analyze the binding of AIMP1 to purified GST-gp96 fragments.

FIG. 17 shows the results of Western blot analysis conducted to analyze whether a gp96 mutant (E791Δ) binds to AIMP1.

FIG. 18 is a schematic diagram showing the functional domain of each of AIMP1 and gp96.

FIG. 19 is a graphic diagram showing the levels of AIMP1 in sera, isolated from normal persons and SLE patients.

### MODE FOR INVENTION

Hereinafter, the present invention will be described in further detail with reference to examples. It is to be understood, however, that these examples are illustrative only, and the scope of the present invention is not limited thereto.

### Example 1

### Identification of gp96 as AIMP1-Binding Protein

### <1-1> Purification of AIMP1-Binding Proteins by Affinity

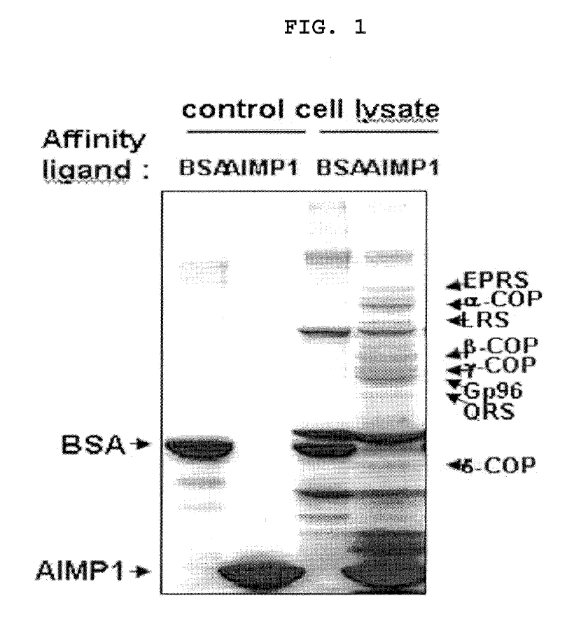

AIMP1 affinity purification was performed to isolate a protein binding to AIMP1, and the protein co-purified with AIMP1 was identified by mass spectrometry. As a result, it was found that gp96 was bound to the AIMP1 protein. Specifically, a recombinant AIMP1 protein and BSA were conjugated to biotin using sulfo-biotin reagent according to the manufacturer's instruction (Pierce). The mouse pancreas was homogenized in a 1% Triton X-100-containing homogenization buffer (25 mM Tris, pH 7.4, 10 mM NaCl, 0.5 mM EDTA, 0.5 mM phenylmethylsulfonyl fluoride, 1 μg/ml leupeptin, 1 μg/ml pepstatin A, and 5 μg/ml aprotinin). The biotin-conjugated AIMP1 and BSA were immobilized on streptavidin beads, and the beads were cultured with 10 mg of protein extract at 4° C. for 12 hours. After washing, the co-precipitated protein was subjected to SDS-PAGE to separate the main band, which was then treated with trypsin (Roche Molecular Biochemicals) at 37° C. for 6 hours. The trypsin-treated peptide fragment was analyzed using a Voyager DE time-of-flight mass spectrometer (Perceptive Biosystems, Inc., Framingham, Mass.), and the analysis results are shown in FIG. 1.

As shown in FIG. 1, it was observed that gp96, tRNA synthases (EPRS, LRS and QRS), known to form complexes with AIMP1, and COPI complex subunits, were proteins binding to AIMP1.

### <1-2> Western Blotting

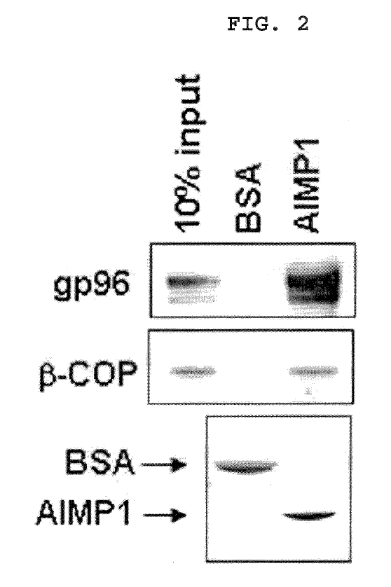

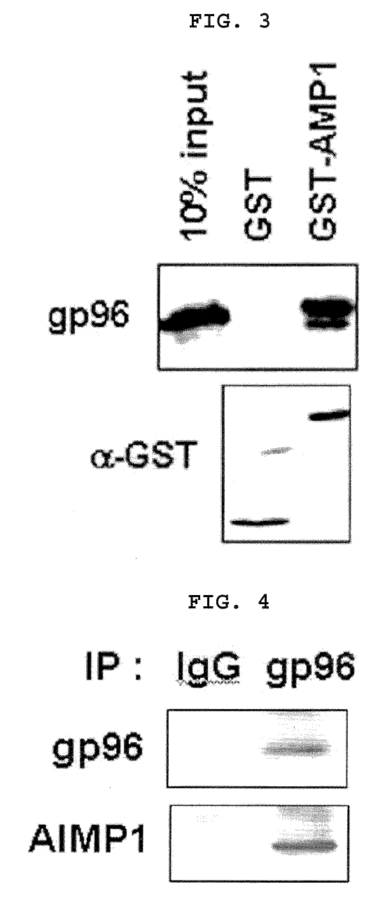

In order to confirm again the binding of AIMP1 to gp96 or β-COP, the protein extracted from the mouse pancreas was purified with the biotin-conjugated AIIM1, isolated according to the method of Example <1-1>, and BSA. The purified protein was analyzed by Western blot using rabbit anti-gp96 antibody (Santa Crus, Calif.) and β-COP antibody, and the analysis results are shown in FIG. 2. Also, the protein extracted from the mouse pancreas was purified with GST or GST-AIMP1 by SDS-PAGE, and the purified protein was analyzed by Western blot using rabbit anti-gp96 antibody (Santa Cruz, Calif.) and mouse anti-GST antibody (Santa Cruz, Calif.). The analysis results are shown in FIG. 3.

As shown in FIGS. 2 and 3, it could be seen that AIMP1 was bound directly to gp96.

### <1-3> Co-Immunoprecipitation

HL-60 cells (American Type Culture Collection, Manassas, Va.), transfected with an AIMP1-encoding plasmid (Ko Y G, et. al., J Biol. Chem., 22; 276(25):23028-33, 2001), were lysed in a lysis buffer (25 mM Tris-HCl, pH 7.4, 10 mM NaCl, 10% glycerol, 1 mM EDTA, 0.5% Triton X-100, 2 mM DTT, 1 mM PMSF and aprotinin). The lysed cells were disrupted with an ultrasonic disrupter for 5 seconds and centrifuged at 14,000 rpm for 15 minutes. The supernatant was collected and used as a protein extract. The extracted protein was mixed with rabbit anti-gp96 antibody (Santa Cruz, Calif.), previously bound to protein A agarose, and then the precipitated protein was immunoprecipitated with rabbit anti-gp96 antibody (Santa Cruz, Calif.) and anti-AIMP1 antibody (Park S. G., et al., J. Biol. Chem. 274:16673-16676, 1999). As a result, it could be seen that AIMP1 and gp96 were co-immunoprecipitated (FIG. 4).

The above results suggest that gp96 binds to AIMP1.

### Example 2

### Examination of Intracellular Location of gp96 by Binding to AIMP1

### <2-1> Intracellular Location of gp96 by AIMP1

The present inventors examined the intracellular location of gp96 in AIMP1^{+/+} and AIMP1^{-/-} MEFs. The AIMP1^{-/-} mice were prepared using a gene trap method (Cecconi, F. & Meyer, B. I., FEBS Lett., 480:63-71, 2000). For this purpose, the genomic DNA of SvEvBrd mice (Lexicon Genetics, USA) was mutated using the gene trap vector VICTR20 (Lexicon Genetics, USA). The mutated genomic DNA was introduced in embryonic stem cells, derived from 129/SvEvBrd mice, and a mutant library was then constructed. From the library, a clone containing an AIMP1 gene, disrupted by the introduction of the gene trap vector, was screened, and the screened clone was named “OST58507”. Then, heterozygous C57/BL6 mice (Samtako) were prepared using the clone according to the protocol of the manufacturer (Lexicon Genetics). The heterozygous mice were mated, thus obtaining 145 wild-type mice (AIMP1^{+/+}), 323 heterozygous mutant mice (AIMP^{+/-}) and 59 homozygous mutant mice (AIMP1^{-/-}).

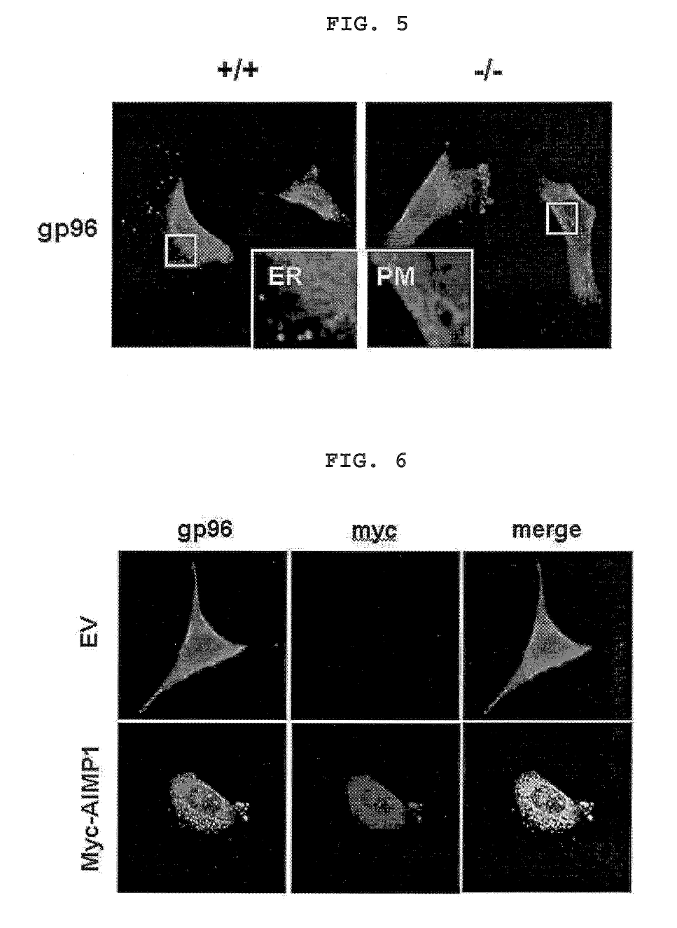

AIMP1^{+/+} and AIMP1^{-/-} MEFs were obtained from 12.5-day-old embryos according to the method described in the literature (Park S G, et. al., Am J. Pathol., 166(2):387-98, 2005). MEF cells were washed with 1×PBS solution and fixed with 100% methanol solution for 5 minutes. The fixed cells were washed again with 1×PBS solution, and anti-gp96 antibody, diluted at 1/100 in a PBS solution containing 1% CAS-1, was allowed to react with the cells. After the cells were washed again with 1×PBS solution, the cells were allowed to react with an FITC (green)-conjugated secondary antibody (green), and the locations of gp96 in AIMP1^{+/+} and AIMP1^{-/-} MEFs were analyzed. The analysis results are shown in FIG. 2a. It was found that, in the MEFs of the wild-type mice, gp96 was located mainly in the ER around the nucleus, and in the MEFs of the AIMP1-deleted mice, gp96 was located in the plasma membrane (see FIG. 5).

Also, a vector comprising myc-tagged AIMP1 was transfected into the MEFs of the AIMP1-deleted mice using Lipofectamine2000 (Invitrogen). The transfected cells were allowed to react with rabbit anti-gp96 antibody (Santa Cruz. CA) or anti-myc antibody (9E10) (Santa Cruz, Calif.) and allowed to react with an FITC (green)- and TRITC (red)-conjugated secondary antibody. The analysis results are shown in FIG. 6.

It was shown that when AIMP1 was overexpressed in the AIMP1-deleted mice, gp96 was located again in ER (FIG. 6). These results suggest that the intracellular location of gp96 is regulated by AIMP1.

### <2-2> Examination of Cell Surface Expression Level gp96 by AIMP1

Generally, gp96 is the ER-resident member of the HSP90 family (Li Z, Dai J, et. al., Front. Biosci, 7:d731-751, 2002). However, it is known that gp96 is expressed on the cell surface in apoptotic or infectious conditions (Basu, S., et. al., Int. Immunol. 12:1539-1546, 2000; Hilf, N. et al., Blood 99: 3676-3682, 2002; Banerjee, P. P. et al., J. Immunol., 169: 3507-3518, 2002).

Because it was found in Example <2-1> that the intracellular location of gp96 was regulated by AIMP1, the present inventors examined whether the cell surface expression level of gp96 is also regulated by AIMP1.

a. Analysis of Cell Surface Expression Level of gp96 in MEF Cells

MEF cells, isolated according to the same method as described in Example <2-1>, were washed with 1×PBS, and then suspended in FACS buffer solution (1×PBS containing 2% FBS, 1% BSA, and 0.1% sodium azide). Then, the cells were pretreated with a general goat antibody. The MEF cells were washed with 1×PBS solution and incubated in FACS buffer solution for 30 minutes to prevent the non-specific binding of an antibody. Then, anti-gp96 antibody was diluted at 1/100 in FACS buffer solution and allowed to react with the cells for 30 minutes. Then, the cells were washed with 1×PBS solution and allowed to react with a secondary antibody, diluted at 1/200 in FACS buffer solution. Then, the cells were analyzed by FACS.

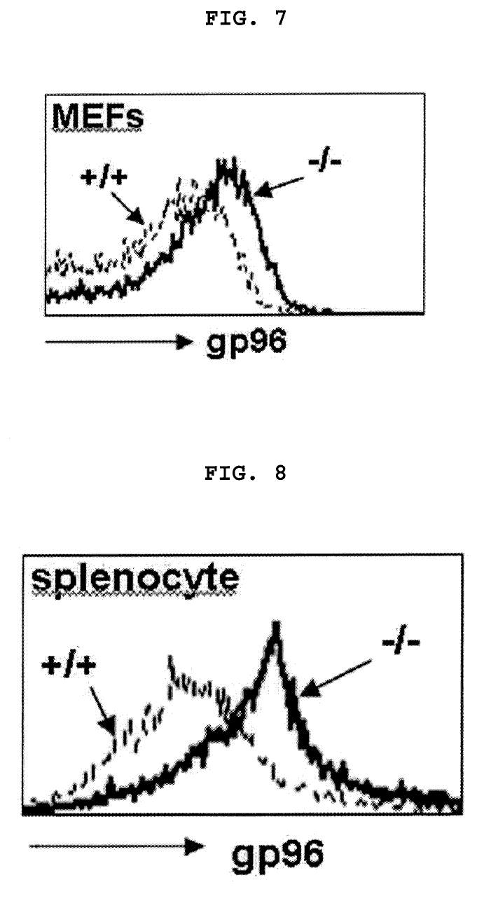

As a result, it could be seen that the cell surface expression level of gp96 in the MEFs in the AIMP1-deleted mice was increased compared to the cell surface expression level of gp96 in the MEFs of the wild-type mice (see FIG. 7).

b. Analysis of Cell Surface Expression Level of gp96 in Spleen Cells

From 12-week-old mice prepared according to the same method as described in Example <2-1>, spleens were isolated, and the spleen cells were suspended in 1×PBS using a cell strainer (Becton Dickinson). The suspended cells were washed, and then re-suspended in 1×PBS.

The results of FACS for the cell surface expression level of gp96 in the splenocytes, isolated from AIMP1^{+/+} and AIMP1^{-/-}, are shown in FIG. 8.

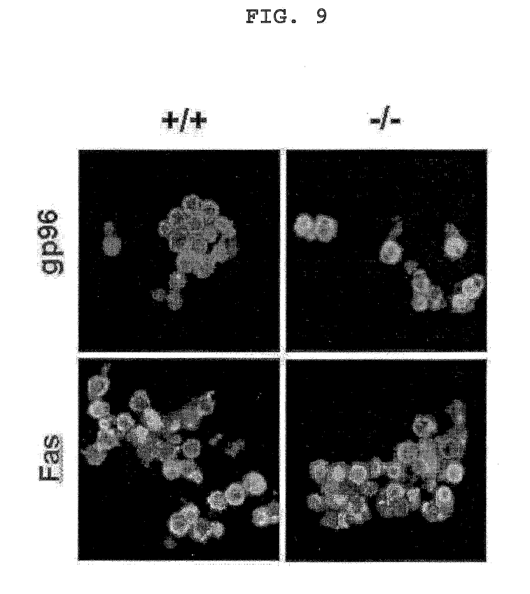

Also, to analyze the cell surface expression level of gp96 in the splenocytes, the cells were immunofluorescence-stained with polyclonal antibody gp96 or anti-Fas antibody, and the nuclei were stained with PI (propidium iodide, red). The stained cells were observed with an immunofluorescent microscope, and the observation results are shown in FIG. 9. Herein, Fas was used as a cell surface marker.

In the analysis results, the cell surface expression level of gp96 was higher in the AIMP1^{-/-} splenocytes than in the AIMP1^{+/+} splenocytes, whereas there was no difference in the cell surface expression level of Fas between the AIMP1^{-/-} splenocytes and the AIMP1^{+/+} splenocytes (see FIGS. 8 and 9).

c. Analysis of Cell Surface Expression Level of gp96 in HeLa Cells Upon Inhibition of Intrinsic AIMP1

HeLa cells (ATCC) were plated on a 6-well plate, and when the cells reached a confluence of 50%, the cells were transfected with an AIMP1 siRNA duplex (Invitrogen, Carlsbad, Calif.) of SEQ ID NO: 15 to a final concentration of 50 nM using Lipofectamine 2000 (Invitrogen, Carlsbad, Calif.) according to the manufacturer's instruction. At 48 hours after the transfection, intrinsic AIMP1 was reduced to the largest extent without influencing cell viability. As a control group, HeLa cells not treated with AIMP1 siRNA were used.

The cell surface expression level of gp96 was analyzed by FACS in the same manner as described in Example <2-2> b, and the analysis results are shown in the left side of FIG. 10. Also, it was analyzed by Western blot in the same manner as described in Example 1, and the analysis results are shown in the right side of FIG. 10.

From the analysis results, it could be seen that, when intrinsic AIMP1 was inhibited using siRNA in HeLa cells, the cell surface expression level of gp96 was increased.

d. Analysis of Cell Surface Expression Level of gp96 in 293 Cells Upon Overexpression of AIMP1

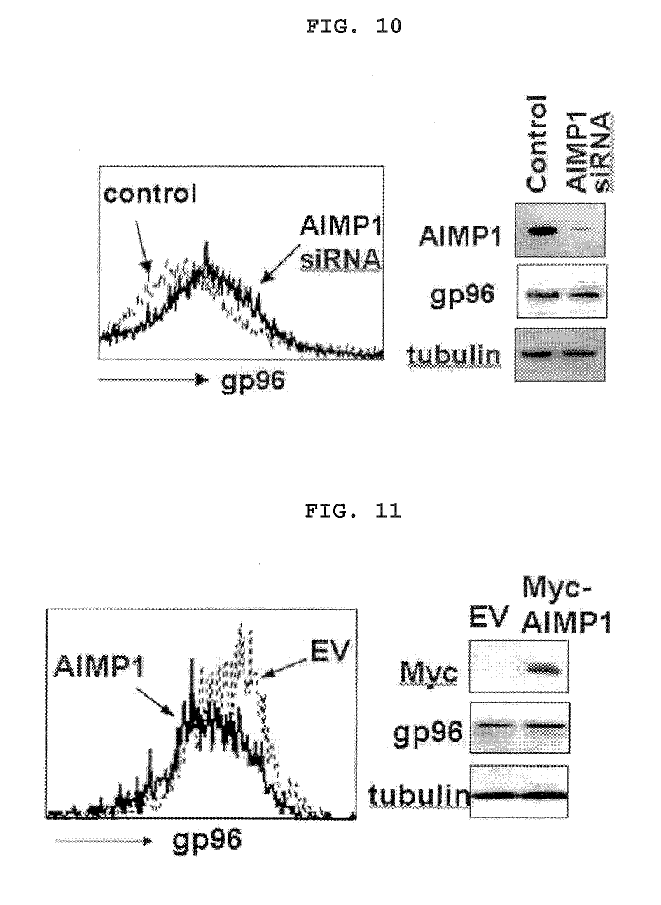

293 cells (ATCC) were transfected with an AIMP1-containing vector or an empty vector, and then the cell surface expression level of gp96 was analyzed by FACS in the same manner as described in Example <2-2> b. The analysis results are shown in the left side of FIG. 11.

Also, the cell surface expression level of gp96 was analyzed by Western blot using an anti-Myc antibody and an anti-gp96 antibody in the same manner as described in Example 1, and the analysis results are shown in the right side of FIG. 11.

From the analysis results, it could be seen that, when AIMP1 was overexpressed, the cell surface expression level of gp96 was decreased.

That is, it could be seen that when AIMP1 was intrinsically inhibited, the cell surface expression level of gp96 was increased, and when AIMP1 was overexpressed in the cells, the cell surface expression level of gp96 was decreased, suggesting the cell surface expression level of gp96 was regulated by AIMP1.

### Example 3

### Examination of Autoimmune Disease Phenotype of AIMP1-Deleted Mice

It was reported that transgenic mice expressing gp96 on the cell surface were prepared, the dendritic cells of the mice were excessively activated, and autoimmune diseases occurred in the mice (Liu B, et. al., Proc Natl. Acad. Sci. USA, 100:15824-15829, 2003).

Because it was found in Example 2 that the cell surface expression level of gp96 was regulated by AIMP1, the present inventors examined whether autoimmune diseases occur in AIMP1-deleted mice as in the mice transfected with gp96.

### <3-1> Production of Autologous Antibody in AIMP1-Deleted Mice

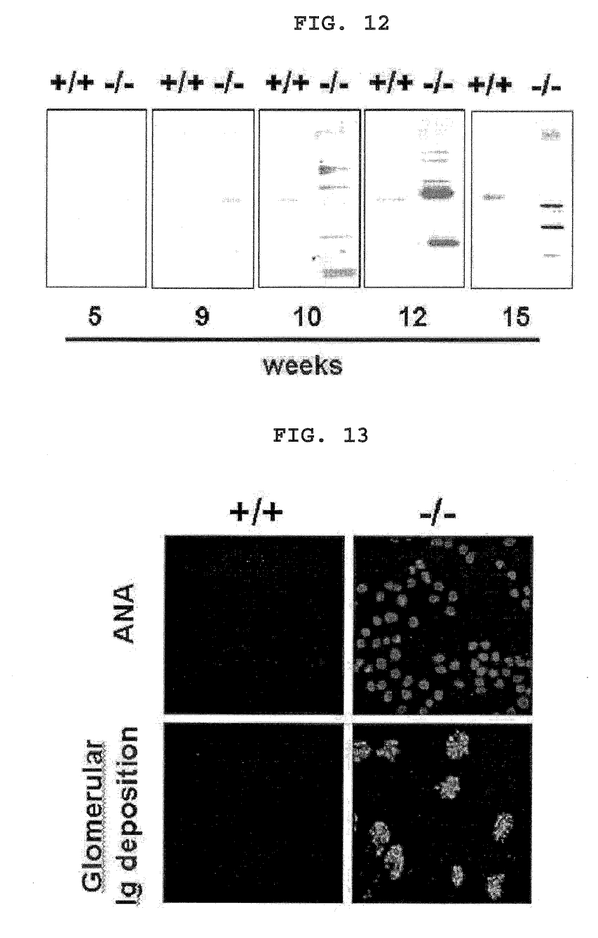

From the blood of the AIMP1^{+/+} and AIMP1^{-/-} mice, prepared according to the method of Example <2-1>, serum was isolated using a clot activator (Becton Dickinson). Also, nuclei were isolated from the livers of 5-week-old, 9-week-old, 10-week-old, 12-week-old and 15-week old mice, and nuclear proteins were separated by SDS-PAGE. In addition, Western blot analysis was performed using autologous serum, and the analysis results are shown in FIG. 12.

As shown in FIG. 12, the nuclear proteins of the AIMP1^{-/-} mice reacted with the autologous sera of mice more than 9 weeks old. Thus, it was shown that, in the AIMP1^{-/-} mice, autoimmune diseases occurred, unlike the case of the wild-type mice.

### <3-2> Production of Anti-Nuclear Antibody in AIMP1-Deleted Mice and Deposition of Immune Complexes in Glomeruli

Whether an antinuclear antibody (ANA) is detected in the sera, isolated according to the method of Example <3-1>, was examined by indirect immunofluorescence using HEP-2-coated slides (INOVA Diagnostics, Inc, San Diego, Calif.). The slides were incubated for 30 minutes with mouse serum, diluted at 1:40 in PBS. After the slides were washed with PBS, FITC-labeled goat anti-mouse Ig (BD Biosciences, Mountain View, Calif.) was added thereto, and then the slides were additionally incubated for 30 minutes. All the experiments were performed in a wet dark room at RT. Then, the slides were washed and mounted with mounting media (Biomeda, Foster City, Calif.). Then, the slides were observed with a fluorescent microscope.

Also, from the mice, the kidneys were extracted using a cryostat, and these low-temperature fragments were blocked with goat serum and then stained with FITC-labeled goat anti-mouse Ig (BD Biosciences, Mountain View, Calif.). Then, the fragments were analyzed with an immunofluorescent microscope.

From the analysis results, it could be observed that an antinuclear antibody (ANA) was present in the sera of the AIMP1^{-/-} mice (upper portion of FIG. 13) and that an immune complex was deposited in the glomeruli of the kidneys (lower portion of FIG. 13).

### <3-3> Production of Hypergammaglobulinaemia in AIMP1-Deleted Mice

The levels of IgA, IgG1, IgG2a, IgG2b, IgG3 and IgM in the serum, isolated from each of 5 wild-type mice, 5 AIMP1^{-/-} mice and 4 AIMP1^{+/-} mice according to the method described in Example <3-1>, were measured using a sandwich ELISA kit (Southern Biotechnology Associates, Birmingham, Ala.). Also, the level of IgE in the serum was measured using an ELISA kit (BD Bioscience, Mountain View, Calif.).

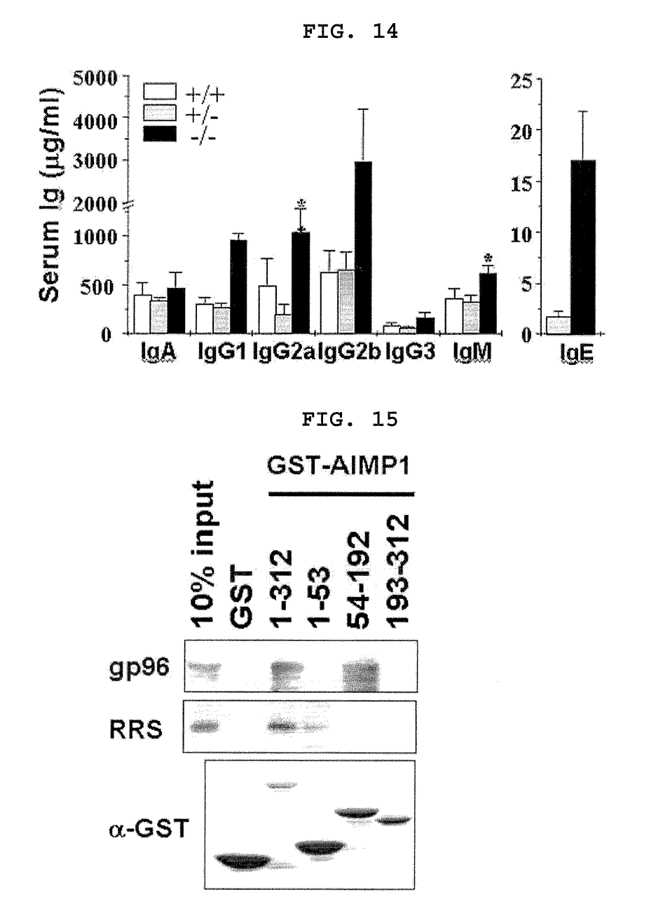

As a result, as shown in FIG. 14, the levels of IgG1, IgG2a, IgM and IgE in the sera of the AIMP1-deleted mice were increased, suggesting that hypergammaglobulinaemia was produced in the mice.

The above results suggest that when AIMP1 is deleted, autoimmune diseases such as lupus occur. That is, it can be seen that AIMP1 binds to gp96 to regulate the cell surface expression level of gp96, and when AIMP1 is deleted, the cell surface expression level of gp96 increases to cause strong immune responses, thus causing autoimmune diseases.

### Example 4

### Identification of Binding Regions of AIMP1 and gp96

### <4-1> Binding of gp96 to AIMP1 or its Fragments

As described above, it was found that AIMP1 was bound to gp96 and that the cell surface expression level of gp96 was regulated by AIMP1. The present inventors identified the binding regions of AIMP1 and gp96.

An AIMP1 protein (SEQ ID NO: 1) consisting of 312 amino acids was prepared according to the method of Park et al. (Park S. G. et al., J. Biol. Chem., 277:45243-45248, 2002).

Each of fragments of AIMP1, that is, fragments of AIMP1-(1-53) (SEQ ID NO: 3), AIMPI-(54-192) (SEQ ID NO: 4) and AIMP1-(193-312) (SEQ ID NO: 6), was prepared.

Each of the fragments was synthesized by PCR using the cDNA of AIMP1 as a template with primer sets specific for each fragment (see Table 1). The PCR reactions were performed in the following conditions: pre-denaturation of template DNA at 95° C. for 2 min; and then 25 cycles at 95° C. for 30 sec, 56° C. for 30 sec and 72° C. for 1 min; followed by final extension at 72° C. for 5 min.

Each of the PCR products and the AIMP1 proteins was digested with EcoRI and XhoI and ligated into a pGEX4T3 vector (Amersham Biosciences), digested with the same enzymes. E. coli BL21 cells were transformed with the vector and cultured to induce the expression of the peptides. Each of the peptides, expressed as GST-tag fusion proteins, was purified on GSH agarose gel. To remove lipopolysaccharide, the protein solution was dialyzed through pyrogen-free buffer (10 mM potassium phosphate buffer, pH 6.0, 100 mM NaCl). After the dialysis, the solution was loaded onto polymyxin resin (Bio-Rad) pre-equilibrated with the same buffer, and then incubated for 20 minutes, followed by elution, thus preparing each of AIMP1 fragments.

TABLE 1
Primer sets used to prepare AIMP1 fragments

| | | |
|---|---|---|
| Primers | Sequences | SEQ ID NO |

| | | |
|---|---|---|
| AIMP1-(1-53) sense | 5′-CGG AAT TCA TGG CAA ATA ATG ATG CTG TTC TGA AG-3′ | 7 |
| AIMP1-(1-53) anti-sense | 5′-GTC TCG AGT TAA GCA TTT TCA ACT CGA AGT TTC-3′ | 8 |
| AIMP1-(54-192) sense | 5′-CGGAATTCAA ACTGAAGAAA GAAATTGAAG AACTG-3′ | 9 |
| AIMP1-(54-192) anti-sense | 5′-GTCTCGAGTT AGCCACTGAC AACTGTCCTT GG-3′ | 10 |
| AIMP1-(193-312) sense | 5′-CGG AAT TCC TGG TGA ATC ATG TTC CTC TTG AAC-3′ | 11 |
| AIMP1-(193-312) anti-sense | 5′-GTC TCG AGT TAT TTG ATT CCA CTG TTG CTC ATG-3′ | 12 |

The purified GST-AIMP1 fragments were cultured with HeLa(ATCC) cell lysates and analyzed by Western blot using a rabbit anti-gp96 antibody (Santa Cruz, Calif.). As a control group, an arginyl-tRNA synthase (RRS) antibody (Jeongwoo Kang, et. al., J. Biol. Chem., 275:31682-31688, 200) was used. The binding assays of the fragments were performed in 25 mM Tris-HCl buffer (containing 120 mM NaCl, 10 mM KCl and 0.5% Triton X-100).

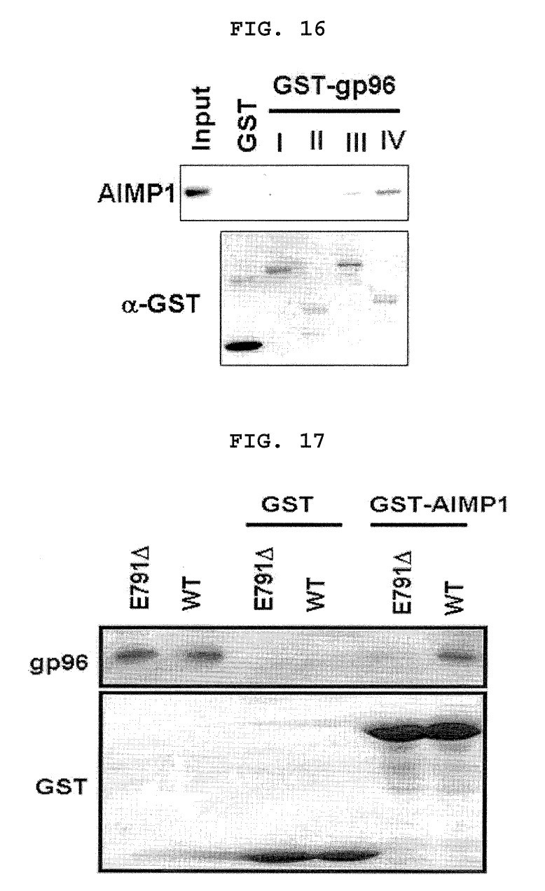

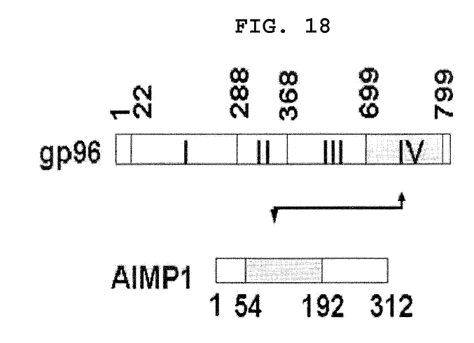

As a result, as shown in FIG. 15, the region of AIMP1, different from the region of AIMP1 binding to RRS used as the control group, was bound to gp96. That is, the region of amino acids 54-192 of AIMP1 was bound to gp96.

### <4-2> Binding of AIMP1 to gp96 Fragments

gp96 is divided into three functional domains. That is, it is known that the region of amino acids 22-287 of gp96 of SEQ ID NO: 11 is responsible for nucleotide/geldanamycin binding, and the region from 288 to 288 368 is an acidic domain (Li Z, Dai J, Zheng H, Liu B, Caudill M: An integrated view of the roles and mechanisms of heat shock protein gp96-peptide complex in eliciting immune response. Front. Biosci 2002, 7: d731-751).

In addition, it is known that the region from 699 to 799 is involved in gp96 oligomerization and self-assembly (Li Z, Dai J, Zheng H, Liu B, Caudill M: An integrated view of the roles and mechanisms of heat shock protein gp96-peptide complex in eliciting immune response. Front. Biosci 2002, 7: d731-751).

Thus, in order to examine what are the effects of the functional domains of gp96 on the binding of AIMP1 to gp96, each of gp96-(22-287; SEQ ID NO: 15), gp96-(288-368; SEQ ID NO: 16), gp96-(369-698; SEQ ID NO: 17) and gp96-(699-799; SEQ ID NO: 18) fragments was prepared.

Each of the fragments was synthesized by PCR amplification using the cDNA of gp96 as a template with a primer set specific for each fragment (Table 2). The PCR reactions were performed in the following conditions: pre-denaturation of template DNA at 95° C. for 2 min; and then 30 cycles of 30 sec at 95° C., 30 sec at 56° C. and 1 min at 72° C.; followed by final extension at 72° C. for 5 min. Each of the PCR products was digested with EcoRI and SalI and ligated into a pGEX4T3 vector (Amersham Biosciences), digested with the same enzymes. E. coli BL21 cells were transformed with the vector and cultured to induce the expression of the peptides. Each of the peptides, expressed as GST-tag fusion proteins, was purified on GSH agarose gel. To remove lipopolysaccharide, the protein solution was dialyzed through pyrogen-free buffer (10 mM potassium phosphate buffer, pH 6.0, 100 mM NaCl). After the dialysis, the solution was loaded onto polymyxin resin (Bio-Rad) pre-equilibrated with the same buffer, and then incubated for 20 minutes, followed by elution, thus preparing each of gp96 fragments.

TABLE 2
Primers sets used to prepare gp96 fragments

| | | |
|---|---|---|
| Primers | Sequences | SEQ ID NO |

| | | |
|---|---|---|
| gp96-(22-287) sense | 5′-GCC GAA TTC GAT GGA CGA TGA AGT TGA TGT GGA TGG-3′ | 20 |
| gp96-(22-287) anti-sense | 5′-CTT GTC GAC TTA TTC AGT CTT GCT GCT CCA TAC-3′ | 21 |
| gp96-(288-368) sense | 5′GCC GAA TTC GAT GAC TGT TGA GGA GCC CAT GGA GG-3′ | 22 |
| gp96-(288-388) anti-sense | 5′-CTT GTC GAC TTA GTC ATC ACT TTC CTT TGA AAA TGA TTG-3′ | 23 |
| gp96-(369-698) sense | 5′-GCC GAA TTC GAT GCC CAT GGC TTA TAT TCA CTT TAC TG-3′ | 24 |
| gp96-(369-698) anti-sense | 5′-CTT GTC GAC TTA CAT GTC TCT GAT CAG CGG GTG-3′ | 25 |
| gp96-(699-799) sense | 5′-GCC GAA TTC GAT GCT TCG ACG AAT TAA GGA AGA TGA AG-3′ | 26 |
| gp96-(699-799) anti-sense | 5′-CTT GTC GAC TTA TTC AGC TGT AGA TTC CTT TGC TG-3′ | 27 |

The purified GST-gp96 fragments were cultured with AIMP1 and analyzed by Western blot using an anti-AIMP1 antibody, and the analysis results are shown in FIG. 16.

As a result, it was shown that the gp96-(699-799; SEQ ID NO: 18), that is, the domain involved in oligomerization, was bound to AIMP1.

These results suggest that the region of amino acids 54-192 of AIMP1, set forth in SEQ ID NO: 4, binds to the region of amino acids 699-799 of gp96, set forth in SEQ ID NO: 18.

### <4-3> Examination of Binding of AIMP1 to gp96 Mutant

The present inventors have found in Example <2-2> that the region of amino acids 54-192 of AIMP1 having an amino acid sequence of SEQ ID NO: 1 binds to the region of amino acids 699-799 of gp96 having an amino acid sequence of SEQ ID NO: 13. To further demonstrate this finding, analysis was performed to examine whether, among mutants recorded in the Genbank, E791 (E791Δ) mutant, which is SNP having mutation in one amino acid of the region of amino acids 699-799 of gp96, which binds to AIMP1, binds to AIMP1. To examine whether the E791 (E791Δ) mutant binds to AIMP1, each of a wild-type gp96-(288-799) fragment and a mutant gp96-(288-799, E791Δ) fragment was prepared.

Each of the fragments was synthesized by PCR amplification using the cDNA of gp96 as a template with a primer set specific for each fragment (Table 3). The PCR reactions were performed in the following conditions: pre-denaturation of template DNA at 95° C. for 2 min; and then 30 cycles of 30 sec at 95° C., 30 sec at 56° C. and 2 min at 72° C.; followed by final extension at 72° C. for 5 min.

Each of the PCR products was digested with EcoRI and SalI and ligated into a pET28c vector (Novagen), digested with the same enzymes. E. coli BL21 cells were transformed with the vector and cultured to induce the expression of the peptides. Each of the peptides, expressed as His-tag fusion proteins, was purified with a nickel column. To remove lipopolysaccharide, the protein solution was dialyzed through pyrogen-free buffer (10 mM potassium phosphate buffer, pH 6.0, 100 mM NaCl). After the dialysis, the solution was loaded onto polymyxin resin (Bio-Rad) pre-equilibrated with the same buffer, and then incubated for 20 minutes, followed by elution, thus preparing each of gp96 fragments.

TABLE 3
Primer sets used to prepare gp96 fragments

| | | |
|---|---|---|
| Primers | Sequences | SEQ ID NO |

| | | |
|---|---|---|
| gp96-(288-799) sense | 5′-GCC GAA TTC GAT GGA CGA TGA AGT TGA TGT GGA TGG-3′ | 28 |
| | GGA TGG-3′ | |
| gp96-(288-799) anti-sense | 5′-CTT GTC GAC TTA TTC AGC TGT AGA TTC CTT | 29 |
| | TGC TG-3′ | |
| gp96-(E791Δ) anti-sense | 5′-CTT gTC gAC TTA TTC AgC TgT AgA TTC CTT TgC | 30 |
| | TgT TTC TTC TTC ATC TgT TCC CAC ATC CAT TTC | |
| | TTC ATC-3′ | |

The purified gp96 proteins were cultured with GST or GST-AIMP1, and then co-immunoprecipitated with a rabbit anti-gp96 antibody (Santa Cruz. CA), and the analysis results are shown in FIG. 17. As shown in FIG. 17, the affinity of the E791 (E791Δ) mutant for AIMP1 was significantly reduced compared to that of the wild type gp96. This suggests that the region of amino acids 699-799 of gp96 is important in the binding of gp96 to AIMP1.

### Example 5

### Measurement of Level of AIMP1 in Blood of Autoimmune Disease Patients

Because it was found in Example 3 that autoimmune diseases occurred in the AIMP1-deleted mice, the present inventors examined the level of AIMP1 in the blood samples of autoimmune disease patients.

Blood samples were collected from 158 systemic lupus erythemasus (SLE) patients and 99 normal persons, and the levels of the AIMP1 protein in the blood samples were measured by an ELISA method. A monoclonal antibody to AIMP1, recognizing the N-terminus of AIMP1, and a monoclonal antibody to AIMP1, recognizing the N-terminus of AIMP1, were prepared in the following manner. 100 μg of an AIMP1 protein antigen was injected intraperitoneally into each of mice. To enlarge the B cell clone, the mice were immunized at 3-4 times at an interval of about one month, and at 3 days after the final immunization, the mice were scarified, and spleens were extracted from the mice. The spleen cells were well mixed with myeloma cells, and 50% PEG1000 (polyethyleneglycol, molecular weight: 1000) was added thereto to cell fusion, thus making hybridomas. After the cell fusion, PEG was washed out with culture medium, and then the cells were suspended in HAT culture medium. The suspension was uniformly dispensed in a 96-well plate. Herein, positive clones (clones specifically the N terminus and C terminus of AIMP1) were selected and cultured. Then, the cultured cells were injected intraperitoneally into mice. After about 10 days, about 5-6 ml of ascites were collected from the mice, and a monoclonal antibody to AIMP1, recognizing the N-terminus of AIMP1, and a monoclonal antibody to AIMP1, recognizing the C-terminus of AIMP1, were purified from the ascites.

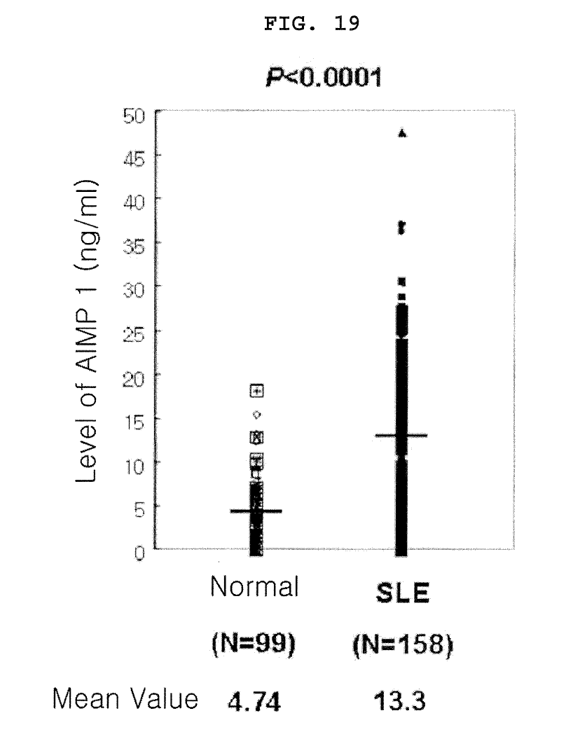

The above-prepared monoclonal antibody recognizing the N-terminus of AIMP1 was dissolved in PBS buffer (pH 7.4) and coated on a 96-well plate (Maxisorp., F96; Nunc) at a concentration of 200 ng/well. After washing, the plate was allowed to react with blocking buffer (PBS buffer containing 1% BSA (bovine serum albumin)) for 1 hour. Serum was isolated from each of the above-collected blood samples and placed in each well of the plate, and 1×PBS containing 1% BSA was added to each well to a final volume of 100 μl. After incubation for 2 hours, the plate was washed and incubated with an HRP-conjugated monoclonal antibody to AIMP1, recognizing the C-terminus of AIMP1. The plate was washed, a substrate reaction solution was added to each well of the plate, and the absorbance at 450 nm was measured. In addition, absorbance values were measured using an ELISA method at concentrations of purified AIMP1 protein of 0, 0.31, 0.63, 1.25, 2.5, 5, 10 and 20 ng/ml, and based on the measured absorbance values, the levels of the AIMP1 protein in the sera were determined. The analysis results are shown in FIG. 19.

As described above, AIMP1 binds to gp96 to regulate the intracellular location of gp96, and as a result, the amount of gp96 present on the cell surface and the resulting immune response are regulated. It was previously found in animal tests that when gp96 was excessively exposed to the cell surface, an autoimmune disease was induced, and it is expected that, in the case of autoimmune disease patients, the binding of gp96 to AIMP1 in the cells breaks, so that gp96 is highly expressed on the cell surface, and AIMP1 is secreted out of the cells and present in blood in large amounts. This expectation was also confirmed by the results shown in FIG. 19. That is, it could be seen that the levels of AIMP1 in the SLE patients were higher than the levels of AIMP1 in the normal persons (see FIG. 19). These results suggest that the blood level of AIMP1 can be used as a novel marker capable of diagnosing autoimmune diseases.

### INDUSTRIAL APPLICABILITY

As described above, the present inventors have found for the first time that the region of amino acids 54-192 of AIMP1, shown in SEQ ID NO: 4, binds directly to the region of amino acids 699-799 of gp96, shown in SEQ ID NO: 18, to assist the localization of gp96 in the endoplasmic reticulum (ER) so as to inhibit the migration of gp96 to the cell surface, thus regulating the amount of gp96 present on the cell surface and the resulting immune response. Accordingly, the binding between the region of amino acids 54-192 of AIMP1, shown in SEQ ID NO: 4, and the region of amino acids 699-799 of gp96, shown in SEQ ID NO: 18, can be used to screen an immune modulator, an anticancer agent and an agent for treating autoimmune diseases. Also, when the binding breaks, immune modulation is not achieved to cause autoimmune diseases, and thus the AIMP1-specific antibody, which is used to measure the level of AIMP1, can be used as a novel marker for diagnosing autoimmune diseases.

1. A method for screening an immune modulator, the method comprising the steps of:
(a) contacting a test agent with an isolated polypeptide comprising an amino acid sequence set forth in SEQ ID NO: 4; and
(b) testing whether the test agent binds to the isolated polypeptide.

2. The method of claim 1, further comprising the steps of:
contacting the candidate substance, tested in step (b), with an isolated polypeptide comprising an amino acid sequence set forth in SEQ ID NO: 18; and
testing whether the test agent binds to the isolated polypeptide comprising the amino acid sequence set forth in SEQ ID NO: 18.

3. A method for screening an immune modulator, the method comprising the steps of:
(a) contacting a test agent with a cell or tissue expressing an isolated polypeptide, comprising an amino acid sequence set forth in SEQ ID NO: 4, and an isolated polypeptide, comprising an amino acid sequence set forth in SEQ ID NO: 18; and
(b) detecting a change in the cell surface expression level of gp96 in the cell or tissue contacted with the test agent relative to the cell surface expression level of gp96 in a cell or tissue not contacted with the test agent.

4. A method of claim 3, wherein the cell or tissue is transfected simultaneously with an isolated polynucleotide, comprising a nucleic acid sequence encoding the amino acid sequence set forth in SEQ ID NO: 4, and an isolated polynucleotide, comprising a nucleic acid sequence encoding the amino acid sequence set forth in SEQ ID NO: 18.

5. A method of claim 4, wherein the nucleic acid sequence encoding the amino acid sequence set forth in SEQ ID NO: 4 is set forth in SEQ ID NO: 5.

6. The method of claim 4, wherein the nucleic acid sequence encoding the amino acid sequence set forth in SEQ ID NO: 18 is set forth in SEQ ID NO: 19.

7. A method for screening an anticancer agent, the method comprising the steps of:
(a) contacting a test agent with an isolated polypeptide comprising an amino acid sequence set forth in SEQ ID NO: 4;
(b) testing whether the test agent binds to the isolated polypeptide;
(c) administering the test agent to a cancer cell or a cancer animal model; and
(d) detecting a change in the progression of cancer in the cancer cell or cancer animal model administered with the test agent.

8. A method for screening an anticancer agent, the method comprising the steps of:
(a) contacting a test agent with a cell or tissue expressing a polypeptide comprising an amino acid sequence set forth in SEQ ID NO: 4;
(b) testing whether the cell surface expression level of gp96 in the cell or tissue contacted with the test agent is increased compared to the cell surface expression level of gp96 in a cell not contacted with the test agent;
(c) administering the test agent to a cancer cell or a cancer animal model; and
(d) detecting a change in the progression of cancer in the cancer cell or cancer animal model administered with the test agent.

9. A method for screening an agent for treating autoimmune diseases, the method comprising the steps of:
(a) contacting a test agent with an isolated polypeptide comprising an amino acid sequence set forth in SEQ ID NO: 18;
(b) testing whether the test agent binds to the isolated polypeptide comprising the amino acid sequence set forth in SEQ ID NO: 18;
(c) administering the test agent to an immune cell or an autoimmune disease animal model; and
(d) measuring the degree of immune suppression in the immune cell or autoimmune disease model administered with the test agent.

10. A method for screening an agent for treating autoimmune diseases, the method comprising the steps of:
(a) contacting a test agent with a cell or tissue expressing an isolated polypeptide comprising an amino acid sequence set forth in SEQ ID NO: 18;
(b) testing whether the cell surface expression level of gp96 in the cell or tissue contacted with the test agent is decreased compared to the cell surface expression level of gp96 in a cell not contacted with the test agent;
(c) administering the test agent to an immune cell or an autoimmune disease animal model; and
(d) measuring the degree of immune suppression in the immune cell or autoimmune disease model administered with the test agent.

11. A composition for diagnosing autoimmune diseases, comprising an antibody specific for an AIMP1 protein.

12. A method for diagnosing autoimmune diseases, the method comprising the steps of:
(a) contacting an antibody specific for an AIMP1 protein with a detection sample;
(b) forming an antigen-antibody complex in the detection sample; and
(c) comparing the formation of the antigen-antibody complex with that in a control group.
